(19) United States
(12) Patent Application Publication
(10) Pub. No.: US 2017/0369948 A1
(43) Pub. Date: Dec. 28, 2017

(54) METHODS AND COMPOSITIONS FOR DETECTING COLORECTAL NEOPLASIAS

(71) Applicant: Case Western Reserve University, Cleveland, OH (US)

(72) Inventors: Sanford D. Markowitz, Pepper Pike, OH (US); Joseph E. Willis, Shaker Heights, OH (US); Helen Moinova, Beachwood, OH (US); Thomas LaFramboise, Shaker Heights, OH (US); Omar de la Cruz Cabrera, Chagrin Falls, OH (US); Ryan Fecteau, Cleveland Heights, OH (US)

(73) Assignee: Case Western Reserve University, Cleveland, OH (US)

(21) Appl. No.: 15/540,965

(22) PCT Filed: Dec. 31, 2015
(86) PCT No.: PCT/US15/68252

§ 371 Date: Jun. 29, 2017

Related U.S. Application Data
(60) Provisional application No. 62/099,021, filed on Dec. 31, 2014.

(51) Int. Cl. C12Q 1/68

(52) U.S. Cl. CPC C12Q 1/6886; C12Q 2600/106; C12Q 2600/154

(57) ABSTRACT

The disclosure provides methods for identifying genomic loci that are differentially methylated in colorectal neoplasias. Identification of methylated genomic loci has numerous uses, including for example, to characterize disease risk, to predict responsiveness to therapy, to non-invasively diagnose subjects and to treat subjects determined to have colorectal neoplasias.

### RELATED APPLICATIONS

This application claims the benefit of priority from U.S. provisional application No. 62/099,021, filed Dec. 31, 2014. The disclosure of the foregoing application is hereby incorporated by reference in its entirety.

### FUNDING

Work described herein was supported by grant nos. UO1CA152756; U54CA163060; T32CA059366; and P50CA150964. The United States Government has certain rights in the invention.

### BACKGROUND

In 2015, it is estimated that there will be nearly 150,000 new cases of colon and/or rectal cancer, and that nearly 50,000 deaths will result from colorectal cancer. People are more likely to survive cancer if the disease is diagnosed at an early stage of development, since treatment at that time is more likely to be successful. Early detection depends upon availability of high-quality methods. Such methods are also useful for determining patient prognosis, selecting therapy, monitoring response to therapy and selecting patients for additional therapy. Consequently, there is a need for cancer diagnostic methods that are specific, accurate, minimally invasive, technically simple and inexpensive.

Gastrointestinal cancers affect millions of patients per year. For example, over 15,000 new cases of esophageal cancer were diagnosed in 2010, and there were nearly as many deaths from this cancer alone. Similarly, about 21,000 new cases of stomach cancer were diagnosed in 2010, and over 10,000 deaths resulted from stomach cancer. The occurrence of colorectal cancer (i.e., cancer of the colon or rectum) is even higher. Approximately 40% of individuals with colorectal cancer die. As with other cancers, these rates can be decreased by improved methods for diagnosis. Although methods for detecting colorectal cancer exist, the methods are not ideal. Generally, a combination of endoscopy, isolation of cells (for example, via collection of cells/tissues from a fluid sample or from a tissue sample), and/or imaging technologies are used to identify cancerous cells and tumors. There are also a variety of specific tests conducted for colorectal cancer, but these have limitations. For example, colon cancer may be detected with digital rectal exams (i.e., manual probing of rectum by a physician), which are relatively inexpensive, but are unpleasant and can be inaccurate. Fecal occult blood testing (i.e., detection of blood in stool) is nonspecific because blood in the stool has multiple causes. Colonoscopy and sigmoidoscopy (i.e., direct examination of the colon with a flexible viewing instrument) are both uncomfortable for the patient and expensive. Double-contrast barium enema (i.e., taking X-rays of barium-filled colon) is also an expensive procedure, usually performed by a radiologist.

Because of the disadvantages of existing methods for detecting or treating colorectal neoplasias/cancers, new methods are needed for colorectal neoplasia/cancer diagnosis and therapy.

### SUMMARY OF THE DISCLOSURE

In certain aspects, the present disclosure is based in part on the discovery of particular human genomic DNA regions (also referred to herein as informative loci or patches) in which the cytosines within CpG dinucleotides are differentially methylated in tissues from lower gastrointestinal neoplasias, e.g., colorectal neoplasia and unmethylated in normal human tissues. In some embodiments, the neoplasia is a cancer.

In one embodiment, the method comprises assaying for the presence of differentially methylated genomic loci in a tissue sample or a bodily fluid sample from a subject. Tissue sample may be obtained from biopsies of the lower gastrointestinal tract, including but not limited to the rectum, colon, and terminal ileum. Tissue samples may be obtained as a biopsy, or as a swab or brushing of the lower gastrointestinal tract (e.g., colon), or other organs believed to contain cancerous cells or tissues. Exemplary bodily fluids include blood, serum, plasma, a blood-derived fraction, stool, colonic effluent, or urine. In one embodiment, the method involves methylation-sensitive restriction enzyme(s). In another embodiment, the method involves methylation-specific PCR. In another embodiment, the method involves restriction enzyme/methylation-specific PCR. In yet another embodiment, the method comprises reacting DNA from the sample with a chemical compound that converts non-methylated cytosine bases (also called “conversion-sensitive” cytosines), but not methylated cytosine bases, to a different nucleotide base. In an embodiment, the chemical compound is sodium bisulfite, which converts unmethylated cytosine bases to uracil. The compound-converted DNA is then amplified using a methylation-sensitive polymerase chain reaction (MSP) employing primers that amplify the compound-converted DNA template if cytosine bases within CpG dinucleotides of the DNA from the sample are methylated. Production of a PCR product indicates that the subject has cancer or precancerous adenomas. Alternatively, the compound-converted DNA is amplified by bisulfite specific methylation indifferent PCR primers and methylation of the parental DNA template is inferred by DNA sequence analysis of the bisulfite converted and amplified product. Other methods for assaying for the presence of methylated DNA are known in the art.

In another embodiment, the present invention provides a detection method of prognosis of a neoplasia (e.g., lower gastrointestinal neoplasia such as a colon neoplasia and/or a rectal neoplasia) in a subject known to have or suspected of having neoplasia. In some embodiments, the neoplasia is cancer. Such method comprises assaying for the presence of methylated informative loci in a tissue sample or bodily fluid from the subject. In certain cases, it is expected that detection of methylated informative loci in a blood fraction is indicative of an advanced state of cancer (e.g., lower gastrointestinal cancer such as colorectal cancer). In other cases, detection of methylated informative loci in a tissue or stool derived sample or sample from other bodily fluids may be indicative of a cancer that will respond to therapeutic agents that demethylate DNA or reactivate expression of genes located within methylated informative loci.

In another embodiment, the present invention provides a method of monitoring over time the status of neoplasia (e.g., lower gastrointestinal neoplasia such as colorectal neoplasia) in a subject. In some embodiments, the neoplasia is a cancer (e.g., a colon neoplasia and/or a rectal neoplasia).

In another embodiment, the present invention provides a method of evaluating therapy in a subject having cancer or suspected of having neoplasia (e.g., lower gastrointestinal neoplasia such as colorectal neoplasia). In some embodiments, the neoplasia is a cancer.

The present invention also relates to oligonucleotide primer sequences for use in assays (e.g., methylation-specific PCR assays or HpaII assays) designed to detect the methylation status of the informative methylated genomic loci.

The present invention also provides a method of inhibiting or reducing growth of neoplasia cells (e.g., lower gastrointestinal neoplasia such as colorectal neoplasia). In some embodiments, the neoplasia is a cancer.

In some embodiments, the disclosure provides for a method for detecting colorectal cancer or colorectal neoplasia, comprising: a) obtaining a human sample; and b) assaying said sample for the presence of methylation within a nucleotide sequence spanning one or more of the following chromosomal loci: i) chr6: 163834751-163834941; ii) chr8: 97506516-97506680; iii) chr12: 113494734-113494933; or iv) chr22: 39853180-39853369; wherein methylation of said nucleotide sequence is indicative of colorectal cancer. In some embodiments, the method comprises: a) obtaining a human sample; and b) assaying said sample for the presence of methylation within a nucleotide sequence spanning one or more of the following chromosomal loci: i) chr6: 163834750-163834862; ii) chr8: 97506522-97506632; iii) chr8: 97506528-97506643; iv) chr12: 113494734-113494841; or v) chr22: 39853251-39853365; wherein methylation of said nucleotide sequence is indicative of colorectal cancer.

In some embodiments, the disclosure provides for a method for detecting colorectal cancer, comprising: a) obtaining a human sample; and b) assaying said sample for the presence of DNA methylation by assay in a bisulfite converted DNA for retention of a cytosine base at any of the Y positions present in one or more of the nucleotide sequences having at least 90% identical to the sequence of any one or more of SEQ ID NOs: 101-200, 401-500, 691-780, 1099-1212, 1351-1374, 1423-1446, 1489-1506, 1577-1602, 1637-1644, 1661-1668, 1681-1684, 1705-1712, 1729-1736 or 1747-1748; wherein methylation of said nucleotide sequence is indicative of colorectal cancer. In some embodiments, the sample is assayed for the presence of DNA methylation by assay in a bisulfite converted DNA for retention of a cytosine base at any of the Y positions present in one or more of the nucleotide sequences having at least 90% identical to the sequence of any one or more of SEQ ID NOs: 1637-1644, 1661-1668, 1681-1684, 1705-1712, 1729-1736 or 1747-1748. In some embodiments, the sample is obtained from a subject suspected of having or is known to have colorectal cancer or colorectal neoplasia. In some embodiments, the assay is methylation-specific PCR. In some embodiments, the method further comprises: a) treating DNA from the sample with a compound that converts a non-methylated cytosine base in the DNA to a different base; b) amplifying a region of the compound converted nucleotide sequence with a forward primer and a reverse primer; and c) analyzing the methylation patterns of said nucleotide sequences. In some embodiments, the method further comprises: a) treating DNA from the sample with a compound that converts a non-methylated cytosine base in the DNA to a different base; b) amplifying a region of the compound converted nucleotide sequence with a forward primer and a reverse primer; and c) detecting the presence and/or amount of the amplified product. In some embodiments, the compound used to treat DNA is a bisulfite compound. In some embodiments, wherein the assay comprises using a methylation-specific restriction enzyme. In some embodiments, the methylation-specific restriction enzyme is selected from the group consisting of: HpaII, SmaI, SacII, EagI, BstUI and BssHII. In some embodiments, the sample is a bodily fluid selected from the group consisting of blood, serum, plasma, a blood-derived fraction, stool, urine and a colonic effluent. In some embodiments, the sample is derived from a tissue. In some embodiments, the sample is a biopsy. In some embodiments, the sample is a brushing.

In some embodiments, the disclosure provides for a method of monitoring over time a colorectal cancer comprising: a) detecting the methylation status of one or more of the Y positions present in one or more of the nucleotide sequences having at least 90% identical to the sequence of any one or more of SEQ ID NOs: 101-200, 401-500, 691-780, 1099-1212, 1351-1374, 1423, 1489-1506, 1577-1602, 1637-1644, 1661-1668, 1681-1684, 1705-1712, 1729-1736 or 1747-1748 from a sample from a subject for a first time; and b) detecting the methylation status of the nucleotide sequence in a sample from the same subject at a later time; wherein absence of methylation in the nucleotide sequence taken at a later time and the presence of methylation in the nucleotide sequence taken at the first time is indicative of cancer regression, and wherein presence of methylation in the nucleotide sequence taken at a later time and the absence of methylation in the nucleotide sequence taken at the first time is indicative of cancer progression. In some embodiments, the sample is a bodily fluid selected from the group consisting of blood, serum, plasma, a blood-derived fraction, stool, urine and a colonic effluent. In some embodiments, the sample is derived from tissue.

In some embodiments, the disclosure provides for a method of treating a subject having colorectal cancer or neoplasia, comprising the step of treating the subject with chemotherapy, radiation therapy and/or with cancer resection or neoplasia resection; wherein said subject has been determined to DNA methylation as detected assay in a bisulfite converted DNA for retention of a cytosine base of one or more of the Y positions present in one or more of the nucleotide sequences having at least 90% identical to the sequence of any one or more of SEQ ID NOs: 101-200, 401-500, 691-780, 1099-1212, 1351-1374, 1423-1446, 1489-1506, 1577-1602, 1637-1644, 1661-1668, 1681-1684, 1705-1712, 1729-1736 or 1747-1748.

In some embodiments, the disclosure provides for a bisulfite converted sequences comprising a nucleotide sequence having at least 90% identical to the sequence of any one or more of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750, and the reverse complements thereof, including all unique fragments of these sequences and their reverse complements. In some embodiments, the disclosure provides for a panel of bisulfite converted sequences selected from these sequences. In some embodiments, the panel corresponds to the combination of sequence regions comprising any one or more of the following combinations of sequences: 1) UnUp62 and UnUp229; 2) UnUp62, UnUp100, UnUp106, UnUp177, UnUp207, UnUp229 and UnUp307; 3) UnUp106 and UnUp146; 4) UnUp280 and UnUp307; 5) UnUp254 and UnUp307; 6) UnUp146 and UnUp254; 7) UnUp177 and UnUp307; 8) UnUp146 and UnUp307; 9) UnUp106 and UnUp307; 10) UnUp106, UnUp177 and UnUp307; 11) UnUp106, UnUp254, and UnUp307; 12) UnUp106, UnUp280 and UnUp307; 13) UnUp177, UnUp254 and UnUp307; 14) UnUp177, UnUp280 and UnUp307; 15) UnUp106, UnUp146, UnUp280 and UnUp307; 16) UnUp106, UnUp146, UnUp254 and UnUp307; 17) UnUp146, UnUp177, UnUp254 and UnUp307; or 18) UnUp106, UnUp207 and UnUp307. In some embodiments, the panels correspond to the combination of sequence regions corresponding to UnUp106, UnUp146, UnUp207, and UnUp307. In some embodiments, the panel further comprises the vimentin sequence. In some embodiments, the panel corresponds to the combination of sequence regions corresponding to vimentin and UnUp146.

In some embodiments, the disclosure provides for an oligonucleotide primer or probe that hybridizes to any of the sequences of provided herein. In some embodiments, the oligonucleotide primer or probe comprises a sequence having at least 90% sequence identity to SEQ ID NO: 1759, 1760, 1761 or 1762. In some embodiments, the primers comprise any sequence having at least 90% sequence identity to any one or more of SEQ ID NOs: 1525-1550, 1689-1696 or 1751-1758. In some embodiments, the primers comprise a primer pair of a forward primer and a reverse primer, and wherein the forward primer and reverse primer are used for PCR amplification of any of the bisulfite converted sequences disclosed herein. In some embodiments, the primer pairs correspond to any one or more of the following primer pairs: 1) 1525 and 1537; 2) 1526 and 1538; 3) 1527 and 1539; 4) 1528 and 1540; 5) 1529 and 1541; 6) 1530 and 1542; 7) 1531 and 1543; 8) 1532 and 1544; 9) 1533 and 1545; 10) 1534 and 1546; 11) 1535 and 1547; 12) 1536 and 1548; 13) 1549 and 1550; 14) 1689 and 1693; 15) 1690 and 1694; 16) 1691 and 1695; 17) 1692 and 1696; 18) 1751 and 1755; 19) 1752 and 1756; 20) 1753-1757; 21) 1754-1758; or 22) 1759 and 1760. In some embodiments, the disclosure provides for a panel of primer pairs selected from any of these primer pairs. In some embodiments, the panel corresponds to the combination of primer pairs for amplifying any of the combinations of sequence regions: 1) UnUp62 and UnUp229; 2) UnUp62, UnUp100, UnUp106, UnUp177, UnUp207, UnUp229 and UnUp307; 3) UnUp106 and UnUp146; 4) UnUp280 and UnUp307; 5) UnUp254 and UnUp307; 6) UnUp146 and UnUp254; 7) UnUp177 and UnUp307; 8) UnUp146 and UnUp307; 9) UnUp106 and UnUp307; 10) UnUp106, UnUp177 and UnUp307; 11) UnUp106, UnUp254, and UnUp307; 12) UnUp106, UnUp280 and UnUp307; 13) UnUp177, UnUp254 and UnUp307; 14) UnUp177, UnUp280 and UnUp307; 15) UnUp106, UnUp146, UnUp280 and UnUp307; 16) UnUp106, UnUp146, UnUp254 and UnUp307; 17) UnUp146, UnUp177, UnUp254 and UnUp307; 18) UnUp106, UnUp207 and UnUp307; or 19) UnUp106, UnUp146, UnUp207, and UnUp307. In some embodiments, the panel further comprises the vimentin sequence. In some embodiments, the panel corresponds to the combination of sequence regions corresponding to vimentin and UnUp146.

In some embodiments, the disclosure provides for a method for selecting an individual to undergo a diagnostic procedure to determine the presence of colon neoplasia, colon adenoma, colon cancer, or recurrence of colon cancer within the body, by obtaining a biological sample from an individual, and determining the presence in DNA from that sample of DNA methylation present in any one or more of the nucleotide sequences having at least 90% identical to the sequence of any one or more of SEQ ID NOs: 1-100, 985-1098, 1327-1350, 1551-1576, 1629-1636, 1697-1704, 1721-1728, and 1745-1746. In some embodiments, the disclosure provides for a method for selecting an individual to undergo a diagnostic procedure to determine the presence of colon neoplasia, colon adenoma, colon cancer, or recurrence of colon cancer within the body, by obtaining a biological sample from an individual, and determining the presence in DNA from that sample of DNA methylation as detected assay in a bisulfite converted DNA for retention of a cytosine base present in any one or more of the nucleotide sequences having at least 90% identical to the sequence of any one or more of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750. In some embodiments, the disclosure provides for a method for selecting an individual to undergo a treatment for colon neoplasia, colon adenoma, colon cancer, or recurrence of colon cancer, by obtaining a biological sample from an individual, and determining the presence in DNA from that sample of DNA methylation as detected assay in a bisulfite converted DNA for retention of a cytosine base present in any one or more of the nucleotide sequences having at least 90% identical to the sequence of any one or more of SEQ ID NOs: 1-100, 985-1098, 1327-1350, 1551-1576, 1629-1636, 1697-1704, 1721-1728, and 1745-1746. In some embodiments, the disclosure provides for a method for selecting an individual to undergo a treatment for colon neoplasia, colon adenoma, colon cancer, or recurrence of colon cancer, by obtaining a biological sample from an individual, and determining the presence in DNA from that sample of DNA methylation as detected assay in a bisulfite converted DNA for retention of a cytosine base present in any one or more of the nucleotide sequences having at least 90% identical to the sequence of any one or more of SEQ ID NOs: 101-300, 401-600, 691-870, 1099-1326, 1351-1398, 1423-1470, 1489-1524, 1577-1628, 1637-1652, 1661-1688, 1681-1696, 1705-1720, 1729-1744, or 1747-1750. In some embodiments, the DNA methylation is detected by cutting one of the DNA sequences with a methylation-sensitive restriction enzyme. In some embodiments, the DNA methylation is detected by bisulfite converting of DNA from the sample and detecting the presence of any of the bisulfite converted DNA sequences disclosed herein. In some embodiments, the bisulfite converted sequences are detected using any of: DNA sequencing, next generation sequencing, methylation specific PCR, methylation specific PCR combined with a fluorogenic hybridization probe, real time methylation specific PCR. In some embodiments, the bisulfite converted sequences are detected using PCR amplification employing any of the PCR primers or primer pairs disclosed herein. In some embodiments, the biological sample is a tissue sample. In some embodiments, the biological sample is a body fluid. In some embodiments, the body fluid is blood, saliva, spit, stool, or urine or a colonic lavage.

In some embodiments, the disclosure provides for a method for determining the response of an individual with colorectal cancer to therapy by detection in a body fluid of DNA methylation as detected by assay in a bisulfite converted DNA for retention of a cytosine base in any one or more of the nucleotide sequences having at least 90% identical to the sequence of any one or more of SEQ ID NOs: 1-100, 985-1098, 1327-1350, 1551-1576, 1629-1636, 1697-1704, 1721-1728, and 1745-1746; wherein increasing levels of methylation over time are indicative of disease progression and a need for change to a new therapy, and wherein absence of increase in levels of methylation over time or decrease in levels of methylation over time are indicative that change in therapy is not required. In some embodiments, the disclosure provides for a method for determining the response of an individual with colorectal cancer to therapy by detection in a body fluid of methylation in any one or more of the nucleotide sequences having at least 90% identical to the sequence of any one or more of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750; wherein increasing levels of methylation over time are indicative of disease progression and a need for change to a new therapy, and wherein absence of increase in levels of methylation over time or decrease in levels of methylation over time are indicative that change in therapy is not required. In some embodiments, the DNA methylation is detected by bisulfite converting DNA from a body fluid and detecting the presence of any of the bisulfite converted DNA sequences disclosed herein.

In some embodiments, the disclosure provides for a bisulfite-converted nucleotide sequence comprising a sequence having at least 80%, 85%, 90%, 91%, 92%, 93%, 94%, 95%, 96%, 97%, 98%, 99% or 100% identity to any of the following sequences SEQ ID NO: 1705-1720, 1577-1628, 1729-1744, and 1747-1750. In some embodiments, the sequence comprises a sequence having at least 80%, 85%, 90%, 91%, 92%, 93%, 94%, 95%, 96%, 97%, 98%, 99% or 100% identity to any of the following sequences SEQ ID NO: 1705-1720. In some embodiments, the sequence comprises a sequence having at least 80%, 85%, 90%, 91%, 92%, 93%, 94%, 95%, 96%, 97%, 98%, 99% or 100% identity to 1706, 1710, 1714 or 1718.

In some embodiments, the disclosure provides for a method of treating a subject having a colorectal neoplasia, comprising the step of treating the subject with chemotherapy, radiation therapy and/or with the resection of the neoplasia; and/or with ablation of the neoplasia; wherein said subject has been determined to have methylation in a sequence that is at least 90% identical to the sequence of any one or more of: SEQ ID NOs: 1-100, 985-1098, 1327-1350, 1551-1576, 1629-1636, 1697-1704, 1721-1728, and 1745-1746, or complements or fragments thereof. In some embodiments, the disclosure provides for a method of treating a subject having a colorectal neoplasia, comprising the step of treating the subject with chemotherapy, radiation therapy and/or with the resection of the neoplasia; and/or with ablation of the neoplasia; wherein said subject has been determined to have DNA methylation by assay in a bisulfite converted DNA for retention of a cytosine base of one or more of the Y positions present in one or more of the nucleotide sequences having at least 90% identity to the sequence of any one or more of: SEQ ID NOs: 101-300, 401-600, 691-870, 1099-1326, 1351-1398, 1423-1470, 1489-1524, 1577-1628, 1637-1652, 1661-1688, 1681-1696, 1705-1720, 1729-1744, or 1747-1750.

### BRIEF DESCRIPTION OF THE FIGURES

FIG. 1 shows a chart summarizing specificity and sensitivity of the methylated colon cancer markers Un-up-146 (in windows 1 and 2); Un-up-207; and Un-up-307 for detecting colon cancer tissue versus normal colon tissue.

FIGS. 2A and 2B show analysis of sample detection by methylation in the amplicon of Un-Up_146. DNA from matched pairs of colon cancer tumors and normal colon tissue (N/T pairs) or circulating DNA from plasma of colon cancer patients or normal control individuals (plasma) was bisulfite converted. The amplicon of Un_Up_146 was amplified using bisulfite specific methylation indifferent primers, and then analyzed by bisulfite sequencing using Next Generation Sequencing technology. Graphs show the sensitivity (Sens) for detecting a tumor sample or blood sample from a cancer patient, and the specificity (Sp) for not detecting a normal colon tissue or the blood from a control normal patient. FIG. 2A shows data for normal colon and colon tumors (N/T pairs). FIG. 2B shows data from plasma samples. Curves show the percent of samples detected (sensitivity) or not detected (specificity) when individual DNA reads that are called positive based on detection of methylation (i.e. retention of unconverted cytosine residues) at greater than or equal to the cutoff specified on the X-axis (e.g. 6+ designates a DNA read is termed methylated if greater than or equal to 6 CpG cytosines are detected as methylated in between the amplification primers). Curves show the percent of samples that are detected (sensitivity) or rejected (specificity) based on detecting a greater than or equal to percentage of DNA reads as being methylated (Y-axis).

FIGS. 3A and 3B show comparative performance of assays for methylation of Vimentin (Vim) versus of methylation for Un_Up_146 in the plasma samples of FIGS. 2A and 2B in which both the Vim and the Un_Up_146 amplicons were analyzed by bisulfite specific sequencing as detailed for FIGS. 2A and 2B. In plasma, Vim remained 100% specific at a cutoff of 6+ CpG for calling a DNA read as methylated and 1% methylated reads for calling a sample as methylated. Un_Up_146 remains 100% specific at a cutoff of 6+ CpG for calling a DNA read as methylated and 2% methylated reads for calling a sample as methylated.

FIG. 4 provides a tabular summary of the sensitivity and specificity of assay of plasma samples for Vim methylation and for Un_Up-146 methylation when the markers were analyzed either individually or in combination (and where the combination is positive if either marker was individually positive). Patients were further categorized as having either early stage (“ES”-stage I or stage II) colon cancer, or as having late stage (“LS”—stage III, stage IV, or metastatic recurrence) colon cancer. FIG. 4 also summarizes the numbers of blood samples from early stage colon cancer patients, late stage colon cancer patients, and normal control individuals that were used in each of the analyses of FIGS. 2-4. Blood samples from colon cancer patients with primary disease were obtained prior to surgery.

### DETAILED DESCRIPTION OF THE INVENTION

### I. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Each embodiment of the invention described herein may be taken alone or in combination with one or more other embodiments of the invention.

Throughout this specification, the word “comprise” or variations such as “comprises” or “comprising” will be understood to imply the inclusion of a stated integer or groups of integers but not the exclusion of any other integer or group of integers.

The articles “a” and “an” are used herein to refer to one or to more than one (i.e., to at least one) of the grammatical object of the article. By way of example, “an element” means one element or more than one element.

The terms “adenoma” is used herein to describe any precancerous neoplasia or benign tumor of epithelial tissue, for example, a precancerous neoplasia of the lower gastrointestinal tract.

The term “colon adenoma” and “polyp” are used herein to describe any precancerous neoplasia of the colon.

The term “blood-derived fraction” herein refers to a component or components of whole blood. Whole blood comprises a liquid portion (i.e., plasma) and a solid portion (i.e., blood cells). The liquid and solid portions of blood are each comprised of multiple components; e.g., different proteins in plasma or different cell types in the solid portion. One of these components or a mixture of any of these components is a blood-derived fraction as long as such fraction is missing one or more components found in whole blood.

The term “colon” as used herein is intended to encompass the right colon (including the cecum), the transverse colon, the left colon, and the rectum. “Colon cancer” or “colon neoplasia” may be of any of the foregoing specific colon origin types.

The terms “colorectal cancer” and “colon cancer” are used interchangeably herein to refer to any cancerous neoplasia of the colon (including the rectum, as defined above).

A “brushing” of the colon/rectum, as referred to herein, may be obtained using any of the means known in the art. In some embodiments, a brushing is obtained by contacting the colon/rectum with a brush, a sponge, a balloon, or with any other device or substance that contacts the colon/rectum, and obtains a colonic/rectal sample.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The terms “compound”, “test compound,” “agent”, and “molecule” are used herein interchangeably and are meant to include, but are not limited to, peptides, nucleic acids, carbohydrates, small organic molecules, natural product extract libraries, and any other molecules (including, but not limited to, chemicals, metals, and organometallic compounds).

The term “compound-converted DNA” herein refers to DNA that has been treated or reacted with a chemical compound that converts unmethylated C bases in DNA to a different nucleotide base. For example, one such compound is sodium bisulfite, which converts unmethylated C to U. If DNA that contains conversion-sensitive cytosine is treated with sodium bisulfite, the compound-converted DNA will contain U in place of C. If the DNA which is treated with sodium bisulfite contains only methylcytosine, the compound-converted DNA will not contain uracil in place of the methylcytosine.

The term “de-methylating agent” as used herein refers to agents that restore activity and/or gene expression of target genes silenced by methylation upon treatment with the agent. Examples of such agents include without limitation 5-azacytidine and 5-aza-2′-deoxycytidine.

The term “detection” is used herein to refer to any process of observing a marker, or a change in a marker (such as for example the change in the methylation state of the marker), in a biological sample, whether or not the marker or the change in the marker is actually detected. In other words, the act of probing a sample for a marker or a change in the marker, is a “detection” even if the marker is determined to be not present or below the level of sensitivity. Detection may be a quantitative, semi-quantitative or non-quantitative observation.

The term “differentially methylated nucleotide sequence” refers to a region of a genomic loci that is found to be methylated in a in cancer tissues or cell lines, but not methylated in the normal tissues or cell lines.

“Gastrointestinal neoplasia” refers to neoplasia of the upper and lower gastrointestinal tract. As commonly understood in the art, the upper gastrointestinal tract includes the esophagus, stomach, and duodenum; the lower gastrointestinal tract includes the remainder of the small intestine and all of the large intestine.

The terms “healthy”, “normal,” and “non-neoplastic” are used interchangeably herein to refer to a subject or particular cell or tissue that is devoid (at least to the limit of detection) of a disease condition, such as a neoplasia.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated or “non-homologous” shares less than 40% identity, preferably less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073, 1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda. Md. 20894; Altschul, S., et al., J. Mol. Biol. 215: 403-410 (1990)). The well known Smith Waterman algorithm may also be used to determine identity.

The term “including” is used herein to mean, and is used interchangeably with, the phrase “including but not limited to.”

The term “isolated” as used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state. Any of the nucleic acid sequences disclosed herein may be “isolated.”

The term “methylation-sensitive PCR” (i.e., MSP) herein refers to a polymerase chain reaction in which amplification of the compound-converted template sequence is performed. Two sets of primers are designed for use in MSP. Each set of primers comprises a forward primer and a reverse primer. One set of primers, called methylation-specific primers (see below), will amplify the compound-converted template sequence if C bases in CpG dinucleotides within the DNA are methylated. Another set of primers, called unmethylation-specific primers (see below), will amplify the compound-converted template sequences if C bases in CpG dinucleotides within the DNA are not methylated.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

“Operably linked” when describing the relationship between two DNA regions simply means that they are functionally related to each other. For example, a promoter or other transcriptional regulatory sequence is operably linked to a coding sequence if it controls the transcription of the coding sequence.

The term “or” is used herein to mean, and is used interchangeably with, the term “and/or”, unless context clearly indicates otherwise.

The terms “proteins” and “polypeptides” are used interchangeably herein.

A “sample” includes any material that is obtained or prepared for detection of a molecular marker or a change in a molecular marker such as for example the methylation state, or any material that is contacted with a detection reagent or detection device for the purpose of detecting a molecular marker or a change in the molecular marker.

As used herein, “obtaining a sample” includes directly retrieving a sample from a subject to be assayed, or directly retrieving a sample from a subject to be stored and assayed at a later time. Alternatively, a sample may be obtained via a second party. That is, a sample may be obtained via, e.g., shipment, from another individual who has retrieved the sample, or otherwise obtained the sample.

A “subject” is any organism of interest, generally a mammalian subject, such as a mouse, and preferably a human subject.

As used herein, the term “specifically hybridizes” refers to the ability of a nucleic acid probe/primer of the invention to hybridize to at least 12, 15, 20, 25, 30, 35, 40, 45, 50 or 100 consecutive nucleotides of a target sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is achieved with about 0.2×SSC at 50° C. Further descriptions of stringency are provided below.

As applied to polypeptides, the term “substantial sequence identity” means that two peptide sequences, when optimally aligned such as by the programs GAP or BESTFIT using default gap, share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity is not likely to affect the properties of a protein. Examples include glutamine for asparagine or glutamic acid for aspartic acid.

The term “informative loci”, as used herein, refers to any of the nucleic acid sequences referred to herein that may be associated with an altered methylation pattern in a colon neoplasia as compared to a sample (e.g., a colon tissue sample) from a healthy control subject. In some embodiments, the informative loci are associated with increased methylation in a colon neoplasia as compared to a sample (e.g., a colon tissue sample) from a healthy control subject.

In some instances, any of the nucleotide sequences disclosed herein contains one or more “Y” positions. Cytosine residues that may be methylated or unmethylated, and hence may be bisulfite converted to T (if unmethylated) or remain as a C (if methylated), are designated with a “Y.”

The term “UnUp106” or “Un-Up-106” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1402, 1414, 1697 or 1701, or fragments or reverse complements thereof. In some embodiments, the UnUp106 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1426, 1438, 1705 or 1709, or fragments or reverse complements thereof. In some embodiments, the UnUp106 sequence refers to a bisulfite converted methylated nucleotide sequence comprising a sequence having at least 80%, 85%, 90° %, 91%, 92%, 93%, 94%, 95%, 96%, 97%, 98%, 99% or 100% identity to the sequence of SEQ ID NO: 1450, 1462, 1713 or 1717, or fragments or reverse complements thereof. In some embodiments, the UnUp106 sequence may be amplified using primers comprising the sequence of SEQ ID NOs: 1689 or 1693, or fragments or reverse complements thereof.

The term “UnUp35” or “Un-Up-35” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1399, 1411, 1551 or 1563, or fragments or reverse complements thereof. In some embodiments, the UnUp35 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1423, 1435, 1577 or 1589, or fragments or reverse complements thereof. In some embodiments, the UnUp35 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1447, 1459, 1603 or 1615, or fragments or reverse complements thereof. In some embodiments, the UnUp35 sequence may be amplified using primers comprising the sequence of SEQ ID NOs: 1525 or 1537, or fragments or reverse complements thereof.

The term “UnUp146” or “Un-Up-146” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1403, 1415, 1698 or 1702, or fragments or reverse complements thereof. In some embodiments, the UnUp146 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1427, 1439, 1706 or 1710, or fragments or reverse complements thereof. In some embodiments, the UnUp146 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1451, 1463, 1714 or 1718, or fragments or reverse complements thereof. In some embodiments, the UnUp146 sequence may be amplified using primers comprising the sequence of SEQ ID NOs: 1690 or 1694, or fragments or reverse complements thereof.

The term “UnUp190” or “Un-Up-190” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1405, 1417, 1557 or 1569, or fragments or reverse complements thereof. In some embodiments, the UnUp190 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1429, 1441, 1583 or 1595, or fragments or reverse complements thereof. In some embodiments, the UnUp190 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1453, 1465, 1609 or 1621, or fragments or reverse complements thereof. In some embodiments, the UnUp190 sequence may be amplified using primers comprising the sequence of SEQ ID NOs: 1531 or 1543, or fragments or reverse complements thereof.

The term “UnUp207” or “Un-Up-207” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1406, 1418, 1699 or 1703, or fragments or reverse complements thereof. In some embodiments, the UnUp207 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1430, 1442, 1707 or 1711, or fragments or reverse complements thereof. In some embodiments, the UnUp207 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1454, 1466, 1715 or 1719, or fragments or reverse complements thereof. In some embodiments, the UnUp207 sequence may be amplified using primers comprising the sequence of SEQ ID NOs: 1691 or 1695, or fragments or reverse complements thereof.

The term “UnUp307” or “Un-Up-307” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1410, 1422, 1700 or 1704, or fragments or reverse complements thereof. In some embodiments, the UnUp307 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1434, 1446, 1708 or 1712, or fragments or reverse complements thereof. In some embodiments, the UnUp307 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1458, 1470, 1716 or 1720, or fragments or reverse complements thereof. In some embodiments, the UnUp307 sequence may be amplified using primers comprising the sequence of SEQ ID NOs: 1692 or 1696, or fragments or reverse complements thereof.

The term “UnUp62” or “Un-Up-62” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1400, 1412, 1552 or 1564, or fragments or reverse complements thereof. In some embodiments, the UnUp62 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1424, 1436, 1578 or 1590, or fragments or reverse complements thereof. In some embodiments, the UnUp62 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1448, 1460, 1604 or 1616, or fragments or reverse complements thereof. In some embodiments, the UnUp62 sequence may be amplified using primers comprising the sequence of SEQ ID NOs: 1526 or 1538, or fragments or reverse complements thereof.

The term “UnUp229” or “Un-Up-229” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1407, 1419, 1559 or 1571, or fragments or reverse complements thereof. In some embodiments, the UnUp229 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1431, 1443, 1585 or 1597, or fragments or reverse complements thereof. In some embodiments, the UnUp229 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1455, 1467, 1611 or 1623, or fragments or reverse complements thereof. In some embodiments, the UnUp229 sequence may be amplified using primers comprising the sequence of SEQ ID NOs: 1533 or 1545, or fragments or reverse complements thereof.

The term “UnUp100” or “Un-Up-100” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1401, 1413, 1553 or 1565, or fragments or reverse complements thereof. In some embodiments, the UnUp100 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1425, 1437, 1579 or 1591, or fragments or reverse complements thereof. In some embodiments, the UnUp100 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1449, 1461, 1605 or 1617, or fragments or reverse complements thereof. In some embodiments, the UnUp100 sequence may be amplified using primers comprising the sequence of SEQ ID NOs: 1527 or 1539, or fragments or reverse complements thereof.

The term “UnUp177” or “Un-Up-177” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1404, 1416, 1556 or 1568, or fragments or reverse complements thereof. In some embodiments, the UnUp177 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1428, 1440, 1582 or 1594, or fragments or reverse complements thereof. In some embodiments, the UnUp177 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1452, 1464, 1608 or 1620, or fragments or reverse complements thereof. In some embodiments, the UnUp177 sequence may be amplified using primers comprising the sequence of SEQ ID NOs: 1530 or 1542, or fragments or reverse complements thereof.

The term “UnUp280” or “Un-Up-280” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1409, 1421, 1561 or 1573, or fragments or reverse complements thereof. In some embodiments, the UnUp280 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1433, 1445, 1587 or 1599, or fragments or reverse complements thereof. In some embodiments, the UnUp280 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1457, 1469, 1613 or 1625, or fragments or reverse complements thereof. In some embodiments, the UnUp280 sequence may be amplified using primers comprising the sequence of SEQ ID NOs: 1535 or 1547, or fragments or reverse complements thereof.

The term “UnUp254” or “Un-Up-254” as used herein refers to a nucleotide sequence comprising a sequence having at least 80%, 85%, 90%, 91%, 92%, 93%, 94%, 95%, 96%, 97%, 98%, 99% or 100% identity to the sequence of SEQ ID NO: 1408, 1420, 1560 or 1572, or fragments or reverse complements thereof. In some embodiments, the UnUp254 sequence refers to a bisulfite converted nucleotide sequence comprising a sequence having at least 80%, 85%, 90%, 91%, 92%, 93%, 94%, 95%, 96%, 97%, 98%, 99% or 100% identity to the sequence of SEQ ID NO: 1432, 1444, 1586 or 1598, or fragments or reverse complements thereof. In some embodiments, the UnUp254 sequence refers to a bisulfite converted methylated nucleotide sequence comprising a sequence having at least 80%, 85%, 90%, 91%, 92%, 93%, 94%, 95%, 96%, 97%, 98%, 99% or 100% identity to the sequence of SEQ ID NO: 1456, 1468, 1612 or 1624, or fragments or reverse complements thereof. In some embodiments, the UnUp254 sequence may be amplified using primers comprising the sequence of SEQ ID NOs: 1534 or 1546, or fragments or reverse complements thereof.

In some embodiments, the disclosure provides for vimentin nucleic acid sequences to be assessed in combination with any of the informative loci described herein. In some embodiments, the disclosure provides for a vimentin nucleic acid sequence is methylated and/or that is bisulfite converted. In some embodiments, the vimentin nucleic acid sequence corresponds to the Vimentin (VIM) locus amplified using primers disclosed in Li et al. (Li M, et al. (2009) Sensitive digital quantification of DNA methylation in clinical samples. Nat Biotechnol 27(9):858-863). These primers correspond to SEQ ID NOs: 1761 and 1762. The amplicons amplified using these primers are as follows:

Vimentin amplicon (+) strand (SEQ ID NO: 1763):
tTYGTttTttTAtYGtAGGATGTTYGGYGGttYGGGtAtYGYGAGtYGGt
YGAGtTttAGtYGGAGtTAYGTGAtTAYGTttAttYGtAttTAtAGttTG
GGtAGt
Vimentin amplicon (-) strand (SEQ ID NO: 1764):
GtTGtttAGGtTGTAGGTGYGGGTGGAYGTAGTtAYGTAGtTtYGGtTGG
AGtTYGGtYGGtTYGYGGTGttYGGGtYGtYGAAtATttTGYGGTAGGAG
GAYGAG.

In some embodiments, the vimentin nucleic acid sequence corresponds to a sequence having at least 80%, 85%, 87%, 90%, 91%, 92%, 93%, 94%, 95%, 96%, 97%, 98%, 99% or 100% identity to SEQ ID NOs: 1763 or 1764, or fragments or complements thereof. In some embodiments, the vimentin nucleic acids correspond to any of those disclosed in US published application no. 2010-0209906, which is incorporated herein in its entirety.

### II. Overview

This application is based at least in part on the recognition that differential methylation of particular genomic loci may be indicative of neoplasia of the lower gastrointestinal tract, e.g., colon. The present findings demonstrate that methylation at any of these genomic loci may be a useful biomarker of neoplasia in the lower gastrointestinal tract.

In certain aspects, the disclosure relates to methods for determining whether a patient is likely or unlikely to suffer from a colon neoplasia. A colon neoplasia is any cancerous or precancerous growth located in, or derived from, the colon. The colon is a portion of the intestinal tract that is roughly three feet in length, stretching from the end of the small intestine to the rectum. Viewed in cross section, the colon consists of four distinguishable layers arranged in concentric rings surrounding an interior space, termed the lumen, through which digested materials pass. In order, moving outward from the lumen, the layers are termed the mucosa, the submucosa, the muscularis propria and the subserosa. The mucosa includes the epithelial layer (cells adjacent to the lumen), the basement membrane, the lamina propria and the muscularis mucosae. In general, the “wall” of the colon is intended to refer to the submucosa and the layers outside of the submucosa. The “lining” is the mucosa.

Precancerous colon neoplasias are referred to as adenomas or adenomatous polyps. Adenomas are typically small mushroom-like or wart-like growths on the lining of the colon and do not invade into the wall of the colon. Adenomas may be visualized through a device such as a colonoscope or flexible sigmoidoscope. Several studies have shown that patients who undergo screening for and removal of adenomas have a decreased rate of mortality from colon cancer. For this and other reasons, it is generally accepted that adenomas are an obligate precursor for the vast majority of colon cancers.

When a colon neoplasia invades into the basement membrane of the colon, it is considered a colon cancer, as the term “colon cancer” is used herein. In describing colon cancers, this specification will generally follow the so-called “Dukes” colon cancer staging system. The characteristics that describe a cancer are generally of greater significance than the particular term used to describe a recognizable stage. The most widely used staging systems generally use at least one of the following characteristics for staging: the extent of tumor penetration into the colon wall, with greater penetration generally correlating with a more dangerous tumor; the extent of invasion of the tumor through the colon wall and into other neighboring tissues, with greater invasion generally correlating with a more dangerous tumor; the extent of invasion of the tumor into the regional lymph nodes, with greater invasion generally correlating with a more dangerous tumor; and the extent of metastatic invasion into more distant tissues, such as the liver, with greater metastatic invasion generally correlating with a more dangerous disease state.

“Dukes A” and “Dukes B” colon cancers are neoplasias that have invaded into the wall of the colon but have not spread into other tissues. Dukes A colon cancers are cancers that have not invaded beyond the submucosa. Dukes B colon cancers are subdivided into two groups: Dukes B1 and Dukes B2. “Dukes B1” colon cancers are neoplasias that have invaded up to but not through the muscularis propria. Dukes B2 colon cancers are cancers that have breached completely through the muscularis propria. Over a five year period, patients with Dukes A cancer who receive surgical treatment (i.e., removal of the affected tissue) have a greater than 90% survival rate. Over the same period, patients with Dukes B1 and Dukes B2 cancer receiving surgical treatment have a survival rate of about 85% and 75%, respectively. Dukes A, B1 and B2 cancers are also referred to as T1, T2 and T3-T4 cancers, respectively.

“Dukes C” colon cancers are cancers that have spread to the regional lymph nodes, such as the lymph nodes of the gut. Patients with Dukes C cancer who receive surgical treatment alone have a 35% survival rate over a five year period, but this survival rate is increased to 60% in patients that receive chemotherapy.

“Dukes D” colon cancers are cancers that have metastasized to other organs. The liver is the most common organ in which metastatic colon cancer is found. Patients with Dukes D colon cancer have a survival rate of less than 5% over a five year period, regardless of the treatment regimen.

In general, neoplasia may develop through one of at least three different pathways, termed chromosomal instability, microsatellite instability, and the CpG island methylator phenotype (CIMP). Although there is some overlap, these pathways tend to present somewhat different biological behavior. By understanding the pathway of tumor development, the target genes involved, and the mechanisms underlying the genetic instability, it is possible to implement strategies to detect and treat the different types of neoplasias.

This disclosure is based at least in part on the recognition that certain target genes may be silenced or inactivated by the differential methylation of CpG islands in the 5′ flanking or promoter regions of the target gene. CpG islands are clusters of cytosine-guanosine residues in a DNA sequence, which are prominently represented in the 5-flanking region or promoter region of about half the genes in our genome. In particular, this application is based at least in part on the recognition that differential methylation of particular genomic loci may be indicative of neoplasia of the lower gastrointestinal tract including, but not limited to, colon neoplasia. The present findings demonstrate that methylation at the informative loci identified herein may be useful biomarkers of neoplasia in the lower gastrointestinal tract.

As noted above, early detection of neoplasia of the lower gastrointestinal tract coupled with appropriate intervention, is important for increasing patient survival rates. Present systems for screening for colon neoplasia are deficient for a variety of reasons, including a lack of specificity and/or sensitivity (e.g. Fecal Occult Blood Test, flexible sigmoidoscopy) or a high cost and intensive use of medical resources (e.g., colonoscopy). Alternative systems for detection of colon neoplasia would be useful in a wide range of other clinical circumstances as well. For example, patients who receive surgical and/or pharmaceutical therapy for colon cancer may experience a relapse. It would be advantageous to have an alternative system for determining whether such patients have a recurrent or relapsed neoplasia of the lower gastrointestinal tract. As a further example, an alternative diagnostic system would facilitate monitoring an increase, decrease or persistence of neoplasia of the lower gastrointestinal tract in a patient known to have such a neoplasia. A patient undergoing chemotherapy may be monitored to assess the effectiveness of the therapy.

### III. Methylation of Informative Loci as Disease Biomarkers

The present disclosure relates at least in part to the identification of informative genomic loci whose altered DNA methylation is indicative of the presence of colorectal neoplasias. SEQ ID NOs: 1-100, 985-1098, 1327-1350, 1551-1576, 1629-1636, 1697-1704, 1721-1728, and 1745-1746, correspond to informative loci that were found to be methylated in colorectal cancer. Detection of methylation in certain of these informative genomic loci may be used to select a patient to undergo a diagnostic procedure such as colonoscopy for detection of adenomas, or colorectal cancer. These informative loci may be useful in screening for these conditions. These informative loci may also be useful in surveillance for disease progression or disease recurrence in individuals that have colorectal cancer. Detection of methylation in certain of these informative genomic loci may also be used to select a patient to undergo a diagnostic procedure for or a treatment of colorectal cancer. In some embodiments, detection of methylation of any of these informative genomic loci may be used in combination with an additional diagnostic assay. In some embodiments, detection of methylation status of any of the informative genomic loci described herein may be used in combination with the detection of the methylation status of the vimentin gene. See, e.g., US published application no. 2010-0209906 and Li M, et al. (2009) Sensitive digital quantification of DNA methylation in clinical samples. Nat Biotechnol 27(9):858-863, each of which is incorporated herein in its entirety.

In some embodiments, any of the nucleotide sequences disclosed herein, or fragments or reverse complements thereof, may contain one or more “Y” residues. Cytosine residues that may be methylated or unmethylated. Cytosines hence may be bisulfite converted to T (if unmethylated) or remain as a C (if methylated). The nucleotide position of bisulfite converted bases, that may be T or C, are, in bisulfite converted DNA sequences, designated with a “Y.” In some embodiments, one or more of the Y residues in any of the sequences disclosed herein (or fragments or reverse complements thereof) designates bisulfite conversion of a methylated C. In some embodiments, one or more of the Y residues in any of the sequences disclosed herein (or fragments or reverse complements thereof) designates bisulfite conversion of an unmethylated C. In some instances, any of the nucleotide sequences disclosed herein contain one or more “Y” positions. In some embodiments, a parental nucleotide sequence is fully unmethylated if the sequence comprises a T at every Y position following bisulfite conversion. In some embodiments, a parental nucleotide sequence is fully methylated if the sequence comprises a C at every Y position following bisulfite conversion. In some embodiments, a parental nucleotide sequence is partially methylated if the sequence comprises at least one C at a Y position and at least one T at a Y position of the sequence following bisulfite conversion. In some embodiments, the bisulfite converted sequences disclosed herein comprise at least one C at a Y position and at least one T at a Y position, i.e., the parental sequence is partially methylated.

In some embodiments, an informative loci in a subject is considered “methylated” for the purposes of determining whether or not the subject is prone to developing and/or has developed a colon neoplasia if the loci is at least 10%, 20%, 30%, 40%, 50%, 60%, 70%, 80%, 90%, or 100% methylated. In some embodiments, a DNA sample from a subject is treated with bisulfite, and the resulting bisulfite sequence corresponds to any of the nucleotide sequences disclosed herein comprising a “Y” nucleotide. In some embodiments, if at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the Y residues of the bisulfite-converted sequence have a C, the sequence is considered “methylated” for the purposes of determining whether or not the subject is prone to developing and/or has developed a colon neoplasia. In some embodiments, a DNA sample from a subject is treated with bisulfite, and the resulting bisulfite sequence corresponds to any of the nucleotide sequences disclosed herein comprising a “Y” nucleotide. In some embodiments, if at least 10%, 20%, 30%, 40%, 50%, 60%, 70%, 80%, 90%, or 100% of the Y residues of the bisulfite-converted sequence have a C, the sequence is considered “methylated” for the purposes of determining whether or not the subject is prone to developing and/or has developed a colon neoplasia. The disclosure provides for informative loci that may be used to assess whether a subject (e.g. a human) has or is prone to developing a colon neoplasia.

In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the Y residues in any of the sequences disclosed herein (or fragments or reverse complements thereof) correspond to methylated C residues (that when bisulfite converted generate a C base). In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the Y residues in any of the sequences disclosed herein (or fragments or reverse complements thereof) correspond to unmethylated C residues (that when bisulfite converted generate uracil that gives rise to a T base). In some embodiments, at least 10%, 20%, 30%, 40%, 50%, 60%, 70%, 80%, 90%, or 100% of the Y residues in any of the sequences disclosed herein (or fragments or reverse complements thereof) correspond to methylated C residues. In some embodiments, at least 10%, 20%, 30%, 40%, 50%, 60%, 70%, 80%, 90%, or 100% of the Y residues in any of the sequences disclosed herein (or fragments or reverse complements thereof) correspond to unmethylated C residues. In some embodiments, any of the sequences disclosed herein (or fragments or reverse complements thereof) is bisulfite-converted. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the Y residues in any of the bisulfite-converted sequences disclosed herein (or fragments or reverse complements thereof) correspond to C. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the Y residues in any of the bisulfite-converted sequences disclosed herein (or fragments or reverse complements thereof) correspond to T. In some embodiments, at least 10%, 20%, 30%, 40%, 50%, 60%, 70%, 80%, 90%, or 100% of the Y residues in any of the bisulfite-converted sequences disclosed herein (or fragments or reverse complements thereof) correspond to C residues. In some embodiments, at least 10%, 20%, 30%, 40%, 50%, 60%, 70%, 80%, 90%, or 100% of the Y residues in any of the bisulfite-converted sequences disclosed herein (or fragments or reverse complements thereof) correspond to T residues.

In some embodiments, the informative loci include sequences associated with any one or more of the plus strand DNA sequences having at least 80%, 85%, 87%, 909%, 91%, 92%, 93%, 94%, 95%, 96%, 97%, 98%, 99% or 100% identity to any of SEQ ID NOs: 1-50, 301-350, 601-645, 985-1034, 1085-1091, 1327-1338, 1399-1410, 1471-1479, 1551-1562, 1575, 1629-1632, 1653-1656, 1677-1678, 1697-1700, 1721-1724, or 1745, or fragments or complements thereof. In particular embodiments, the informative loci include sequences associated with any one or more of the plus strand DNA sequences having at least 80%, 85%, 87%, 90%, 91%, 92%, 93%, 94%, 95%, 96%, 97%, 98%, 99% or 100% identity to any of SEQ ID NOs: 1327-1338, 1399-1410, 1471-1479, 1551-1562, 1575, 1629-1632, 1653-1656, 1677-1678, 1697-1700, 1721-1724, or 1745, or fragments or complements thereof. In particular embodiments, the informative loci include sequences associated with any one or more of the plus strand DNA sequences having at least 80%, 85%, 87%, 90%, 91%, 92%, 93%, 94%, 95%, 96%, 97%, 98%, 99% or 100% identity to any of SEQ ID NOs: 1629-1632, 1653-1656, 1677-1678, 1697-1700, 1721-1724, or 1745, or fragments or complements thereof. In some embodiments, the informative loci are associated with increased methylation in a colon neoplasia (e.g., colon cancer), as compared to the same sample types taken from a healthy control subject.

In some embodiments, the informative loci or amplicon of the informative loci are treated with an agent, such as bisulfite. In some embodiments, the informative loci include sequences that have been treated with bisulfite. In some embodiments, the disclosure provides for bisulfite converted sequences of any of the plus DNA strands disclosed herein. In some embodiments, the disclosure provides for bisulfite-treated sequences of any of the plus DNA strands disclosed herein. In some embodiments, the bisulfite-converted plus-strand DNA sequences include any one or more having at least 80%, 85%, 87%, 90%, 91%, 92%, 93%, 94%, 95%, 96%, 97%, 98%, 99% or 100% identity to any of SEQ ID NOs: 101-150, 401-450, 691-735, 1099-1148, 1199-1205, 1351-1362, 1423-1434, 1489-1497, 1577-1588, 1601, 1637-1640, 1661-1664, 1681-1682, 1705-1708, 1729-1732 or 1747, or fragments or complements thereof. In particular embodiments, the bisulfite-converted plus-strand DNA sequences include any one or more having at least 80%, 85%, 87%, 90%, 91%, 92%, 93%, 94%, 95%, 96%, 97%, 98%, 99% or 100% identity to any of SEQ ID NOs: 1351-1362, 1423-1434, 1489-1497, 1577-1588, 1601, 1637-1640, 1661-1664, 1681-1682, 1705-1708, 1729-1732 or 1747, or fragments or complements thereof. In particular embodiments, the bisulfite-converted plus-strand DNA sequences include any one or more having at least 80%, 85%, 87%, 90%, 910%, 92%, 93%, 94%, 95%, 96%, 97%, 98%, 99% or 100% identity to any of SEQ ID NOs: 1637-1640, 1661-1664, 1681-1682, 1705-1708, 1729-1732 or 1747, or fragments or complements thereof.

In some embodiments, the informative loci include methylated nucleic acid sequences that have been treated with bisulfite. In some embodiments, the bisulfite-converted methylated plus-strand DNA sequences have at least 80%, 85%, 87%, 90%, 91%, 92%, 93%, 94%, 95%, 96%, 97%, 98%, 99% or 100% identity to any of SEQ ID NOs: 201-250, 501-550, 781-825, 1213-1262, 1313-1319, 1375-1386, 1447-1458, 1507-1515, 1603-1614, 1627, 1645-1648, 1669-1672, 1685-1686, 1713-1716, 1737-1740, or 1749, or any fragments or complements thereof. In particular embodiments, the bisulfite-converted methylated plus-strand DNA sequences have at least 80%, 85%, 87%, 90%, 91%, 92%, 93%, 94%, 95%, 96%, 97%, 98%, 99% or 100% identity to any of SEQ ID NOs: 1375-1386, 1447-1458, 1507-1515, 1603-1614, 1627, 1645-1648, 1669-1672, 1685-1686, 1713-1716, 1737-1740, or 1749, or fragments or complements thereof. In particular embodiments, the bisulfite-converted methylated plus-strand DNA sequences have at least 80%, 85%, 87%, 90%, 91%, 92%, 93%, 94%, 95%, 96%, 97%, 98%, 99% or 100% identity to any of SEQ ID NOs: 1645-1648, 1669-1672, 1685-1686, 1713-1716, 1737-1740, or 1749, or fragments or complements thereof.

In some embodiments, the informative loci include sequences associated with any one or more of the minus strand DNA sequences having at least 80%, 85%, 87%, 90%, 91%, 92%& 93%, 94%, 95%, 96%, 97%, 98%, 99% or 100% identity to any of SEQ ID NOs: 51-100, 351-400, 646-690, 1033-1084, 1092-1098, 1339-1350, 1411-1422, 1480-1488, 1563-1574, 1576, 1633-1636, 1657-1660, 1679-1680, 1701-1704, 1725-1728 or 1746, or fragments or complements thereof. In particular embodiments, the informative loci include sequences associated with any one or more of the minus strand DNA sequences having at least 80%, 85%, 87%, 90%, 91%, 92%, 93%, 94%, 95%, 96%, 97%, 98%, 99% or 100% identity to any of SEQ ID NOs: 1339-1350, 1411-1422, 1480-1488, 1563-1574, 1576, 1633-1636, 1657-1660, 1679-1680, 1701-1704, 1725-1728 or 1746, or fragments or complements thereof. In particular embodiments, the informative loci include sequences associated with any one or more of the minus strand DNA sequences having at least 80%, 85%, 87%, 90%, 91%, 92%, 93%, 94%, 95%, 96%, 97%, 98%, 99% or 100% identity to any of SEQ ID NOs: 1633-1636, 1657-1660, 1679-1680, 1701-1704, 1725-1728 or 1746, or fragments or complements thereof. In some embodiments, the informative loci are associated with increased methylation in a colon neoplasia (e.g., colon cancer), as compared to the same sample types taken from a healthy control subject.

In some embodiments, the informative loci or amplicon of the informative loci are treated with an agent, such as bisulfite. In some embodiments, the informative loci include sequences that have been treated with bisulfite. In some embodiments, the disclosure provides for bisulfite converted sequences of any of the minus DNA strands disclosed herein. In some embodiments, the disclosure provides for bisulfite-treated sequences of any of the minus DNA strands disclosed herein. In some embodiments, the bisulfite-converted minus-strand DNA sequences include any one or more having at least 80%, 85%, 87%, 90%, 91%, 92%, 93%, 94%, 95%, 96%, 97%, 98%, 99% or 100% identity to any of SEQ ID NOs: 151-200, 451-500, 736-780, 1149-1198, 1206-1212, 1363-1374, 1435-1446, 1498-1506, 1589-1600, 1602, 1641-1644, 1665-1668, 1683-1684, 1709-1712, 1733-1736 or 1748, or fragments or complements thereof. In particular embodiments, the bisulfite-converted minus-strand DNA sequences include any one or more having at least 80%, 85%, 87%, 90%, 91%, 92%, 93%, 94%, 95%, 96%, 97%, 98%, 99% or 100% identity to any of SEQ ID NOs: 1363-1374, 1435-1446, 1498-1506, 1589-1600, 1602, 1641-1644, 1665-1668, 1683-1684, 1709-1712, 1733-1736 or 1748, or fragments or complements thereof. In particular embodiments, the bisulfite-converted minus-strand DNA sequences include any one or more having at least 80%, 85%, 87%, 90%, 91%, 92%, 93%, 94%, 95%, 96%, 97%, 98%, 99% or 100% identity to any of SEQ ID NOs: 1641-1644, 1665-1668, 1683-1684, 1709-1712, 1733-1736 or 1748, or fragments or complements thereof.

In some embodiments, the informative loci include methylated nucleic acid sequences that have been treated with bisulfite. In some embodiments, the bisulfite-converted methylated minus-strand DNA sequences have at least 80%, 85%, 87%, 90%, 91%, 92%, 93%, 94%, 95%, 96%, 97%, 98%, 99% or 100% identity to any of SEQ ID NOs: 251-300, 551-600, 826-870, 1263-1312, 1320-1326, 1387-1398, 1459-1470, 1516-1524, 1615-1626, 1628, 1649-1652, 1673-1676, 1687-1688, 1717-1720, 1741-1744 or 1750, or any fragments or complements thereof. In particular embodiments, the bisulfite-converted methylated minus-strand DNA sequences have at least 80%, 85%, 87%, 90%, 91%, 92%, 93%, 94%, 95%, 96%, 97%, 98%, 99% or 100% identity to any of SEQ ID NOs: 1387-1398, 1459-1470, 1516-1524, 1615-1626, 1628, 1649-1652, 1673-1676, 1687-1688, 1717-1720, 1741-1744 or 1750, or fragments or complements thereof. In particular embodiments, the bisulfite-converted methylated minus-strand DNA sequences have at least 80%, 85%, 87%, 90%, 91%, 92%, 93%, 94%, 95%, 96%, 97%, 98%, 99% or 100% identity to any of SEQ ID NOs: 1649-1652, 1673-1676, 1687-1688, 1717-1720, 1741-1744 or 1750, or fragments or complements thereof.

In particular embodiments, the disclosure provides for nucleic acid sequences corresponding to the UnUp35, UnUp190, UnUp62, UnUp100, UnUp106, UnUp146, UnUp177, UnUp207, UnUp229, UnUp254, UnUp280, and/or UnUp307 sequences, as defined herein. In some embodiments, any of the UnUp35, UnUp190, UnUp62, UnUp100, UnUp106, UnUp146, UnUp177, UnUp207, UnUp229, UnUp254, UnUp280, and/or UnUp307 nucleotide sequences disclosed herein are bisulfite converted. In some embodiments, any of the UnUp35, UnUp190, UnUp62, UnUp100, UnUp106, UnUp146, UnUp177, UnUp207, UnUp229, UnUp254, UnUp280, and/or UnUp307 nucleotide sequences disclosed herein are methylated and bisulfite converted. In some embodiments, any of the UnUp35, UnUp190, UnUp62, UnUp100, UnUp106, UnUp146, UnUp177, UnUp207, UnUp229, UnUp254, UnUp280, and/or UnUp307 nucleotide sequences disclosed herein are sequences that have been amplified from a nucleic acid sequence taken from a sample from a subject. In some embodiments, the nucleic acid sequence has been amplified following bisulfite conversion. In some embodiments, the nucleic acid sequence has been amplified following bisulfite conversion and using methylation specific primers.

In particular embodiments, the disclosure provides for nucleic acid sequences corresponding to the UnUp106, UnUp146, UnUp207 and/or UnUp307, as defined herein. In some embodiments, any of the UnUp106, UnUp146, UnUp207 and/or UnUp307 nucleotide sequences disclosed herein are bisulfite converted. In some embodiments, any of the UnUp106, UnUp146, UnUp207 and/or UnUp307 nucleotide sequences disclosed herein are methylated and bisulfite converted. In some embodiments, any of the UnUp106, UnUp146, UnUp207 and/or UnUp307 nucleotide sequences disclosed herein are sequences that have been amplified from a nucleic acid sequence taken from a sample from a subject. In some embodiments, the nucleic acid sequence has been amplified following bisulfite conversion. In some embodiments, the nucleic acid sequence has been amplified following bisulfite conversion and using methylation specific primers.

The present disclosure contemplates methods for selecting an individual to undergo a diagnostic procedure to determine the presence of colon neoplasia, colon adenoma, colon cancer, or recurrence of colon cancer within the body, by obtaining a biological sample from an individual, and determining in said sample the presence of DNA methylation of any of the sequences having at least 80%, 85%, 87%, 90%, 91%, 92%, 93%, 94%, 95%, 96%, 97%, 98%, 99% or 100% identity to any of SEQ ID NOs: 1-100, 301-400, 601-690, 985-1098, 1327-1350, 1399-1422, 1471-1488, 1551-1576, 1629-1636, 1653-1660, 1677-1680, 1697-1704, 1721-1728, or 1745-1746, or any fragments or complements thereof. The present disclosure contemplates methods for selecting an individual to undergo a diagnostic procedure to determine the presence of colon neoplasia, colon adenoma, colon cancer, or recurrence of colon cancer within the body, by obtaining a biological sample from an individual, and determining in said sample the presence of DNA methylation of at least one of the sequences of any of the sequences of UnUp62, UnUp100, UnUp106, UnUp146, UnUp177, UnUp207, UnUp229, UnUp254, UnUp280, and/or UnUp307, as defined herein.

The present disclosure also contemplates methods for selecting an individual to undergo a treatment for colon neoplasia, colon adenoma, colon cancer, or recurrence of colon cancer, by obtaining a biological sample from an individual, and determining in said sample the presence of DNA methylation in at least one of the sequences of any of the sequences having at least 80%, 85%, 87%, 90%, 91%, 92%, 93%, 94%, 95%, 96%, 97%, 98%, 99% or 100% identity to any of SEQ ID NOs: 1-100, 301-400, 601-690, 985-1098, 1327-1350, 1399-1422, 1471-1488, 1551-1576, 1629-1636, 1653-1660, 1677-1680, 1697-1704, 1721-1728, or 1745-1746, or any fragments or complements thereof. The present disclosure also contemplates methods for selecting an individual to undergo a treatment for colon neoplasia, colon adenoma, colon cancer, or recurrence of colon cancer, by obtaining a biological sample from an individual, and determining in said sample the presence of DNA methylation in at least one of the sequences of any of the sequences of UnUp62, UnUp100, UnUp106, UnUp146, UnUp177, UnUp207, UnUp229, UnUp254, UnUp280, and/or UnUp307, as defined herein.

The present disclosure also contemplates methods for determining the response of an individual with colorectal cancer to therapy by obtaining a biological sample from an individual with colorectal cancer, and determining in said sample the presence of DNA methylation in at least one of the sequences of any of the sequences having at least 80%, 85%, 87%, 90%, 91%, 92%, 93%, 94%, 95%, 96%, 97%, 98%, 99% or 100% identity to any of SEQ ID NOs: 1-100, 301-400, 601-690, 985-1098, 1327-1350, 1399-1422, 1471-1488, 1551-1576, 1629-1636, 1653-1660, 1677-1680, 1697-1704, 1721-1728, or 1745-1746, or any fragments or complements thereof. The present disclosure also contemplates methods for determining the response of an individual with colorectal cancer to therapy by obtaining a biological sample from an individual with colorectal cancer, and determining in said sample the presence of DNA methylation in at least one of the sequences of any of the sequences of UnUp62, UnUp100, UnUp106, UnUp146, UnUp177, UnUp207, UnUp229, UnUp254, UnUp280, and/or UnUp307, as defined herein. In some implementations, an increase in levels of methylation over time is indicative of disease progression and a need for change to a new therapy, whereas an absence of increase in levels of methylation over time or a decrease in levels of methylation over time is indicative that change in therapy is not required.

The present disclosure contemplates methods for selecting an individual to undergo a diagnostic procedure to determine the presence of colon neoplasia, colon adenoma, colon cancer, or recurrence of colon cancer within the body, by obtaining a biological sample from an individual, and determining in said sample the presence of DNA methylation by bisulfite conversion and assay for C bases of any of the sequences having at least 80%, 85%, 87%, 90%, 91%, 92%, 93%, 94%, 95%, 96%, 97%, 98%, 99% or 100% identity to any of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750, or any fragments or complements thereof. The present disclosure contemplates methods for selecting an individual to undergo a diagnostic procedure to determine the presence of colon neoplasia, colon adenoma, colon cancer, or recurrence of colon cancer within the body, by obtaining a biological sample from an individual, and determining in said sample the presence of DNA methylation of at least one of the sequences of any of the sequences of UnUp62, UnUp100, UnUp106, UnUp146, UnUp177, UnUp207, UnUp229, UnUp254, UnUp280, and/or UnUp307, as defined herein.

The present disclosure also contemplates methods for selecting an individual to undergo a treatment for colon neoplasia, colon adenoma, colon cancer, or recurrence of colon cancer, by obtaining a biological sample from an individual, and determining in said sample the presence of DNA methylation by bisulfite conversion and assay for C bases in at least one of the sequences of any of the sequences having at least 80%, 85%, 87%, 90%, 91%, 92%, 93%, 94%, 95%, 96%, 97%, 98%, 99% or 100% identity to any of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750, or any fragments or complements thereof. The present disclosure also contemplates methods for selecting an individual to undergo a treatment for colon neoplasia, colon adenoma, colon cancer, or recurrence of colon cancer, by obtaining a biological sample from an individual, and determining in said sample the presence of DNA methylation in at least one of the sequences of any of the sequences of UnUp62, UnUp100, UnUp106, UnUp146, UnUp177, UnUp207, UnUp229, UnUp254, UnUp280, and/or UnUp307, as defined herein.

The present disclosure also contemplates methods for determining the response of an individual with colorectal cancer to therapy by obtaining a biological sample from an individual with colorectal cancer, and determining in said sample the presence of DNA methylation by bisulfite conversion and assay for C bases in at least one of the sequences of any of the sequences having at least 80%, 85%, 870%, 90%, 91%, 92%, 93%, 94%, 95%, 96%, 97%, 98%, 99% or 100% identity to any of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750, or any fragments or complements thereof. The present disclosure also contemplates methods for determining the response of an individual with colorectal cancer to therapy by obtaining a biological sample from an individual with colorectal cancer, and determining in said sample the presence of DNA methylation in at least one of the sequences of any of the sequences of UnUp62, UnUp100, UnUp106, UnUp146, UnUp177, UnUp207, UnUp229, UnUp254, UnUp280, and/or UnUp307, as defined herein. In some implementations, an increase in levels of methylation over time is indicative of disease progression and a need for change to a new therapy, whereas an absence of increase in levels of methylation over time or a decrease in levels of methylation over time is indicative that change in therapy is not required.

The present disclosure also provides sequences that will hybridize under highly stringent conditions to any of the informative loci disclosed herein, or fragments or complements thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

In other embodiments, the disclosure also provides the methylated forms of any of the informative loci disclosed herein, or fragments or complements thereof, wherein the cytosine bases of the CpG islands present in said sequences are methylated. In other words, the nucleotide sequences listed of any of the sequences disclosed herein may be either in the methylated status (e.g., as seen in neoplasias) or in the unmethylated status (e.g., as seen in normal cells). In further embodiments, the nucleotide sequences of the disclosure can be isolated, recombinant, and/or fused with a heterologous nucleotide sequence, or in a DNA library.

In certain embodiments, the present disclosure provides bisulfite-converted nucleotide sequences, for example, bisulfite-converted sequences of any of the sequences disclosed herein. In some embodiments, the bisulfite-converted nucleotide sequences are any of the sequences having at least 80%, 85%, 87%, 90%, 91%, 92%, 93%, 94%, 95%, 96%, 97%, 98%, 99% or 100% identity to any of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750, or any fragments or reverse complements thereof. In particular embodiments, the bisulfite-converted nucleotide sequences are any of the sequences having at least 80%, 85%, 87%, 90%, 91%, 92%, 93%, 94%, 95%, 96%, 97%, 98%, 99% or 100% identity to any of SEQ ID NOs: 1705-1720, 1578-1579, 1582, 1585-1587, 1590-1591, 1594, 1597-1599, 1604-1605, 1608, 1612-1613, 1616-1617, 1620, and 1624-1625. In some embodiments, the sequence comprises a sequence having at least 80%, 85%, 90%, 91%, 92%, 93%, 94%, 95%, 96%, 97%, 98%, 99% or 100% identity to any of the following sequences SEQ ID NO: 1705-1720. In some embodiments, the sequence comprises a sequence having at least 80% 85%, 90%, 91%, 92%, 93%, 94%, 95%, 96%, 97%, 98%, 99% or 100% identity to 1706, 1710, 1714 or 1718.

Such bisulfite-converted nucleotide sequences can be used for detecting the methylation status, for example, by an MSP reaction or by direct sequencing. In some embodiments, the bisulfite-converted nucleotide sequences are sequenced by means of next-generation sequencing. These bisulfite-converted sequences are also of use for designing primers for MSP reactions that specifically detect methylated or unmethylated nucleotide sequences following bisulfite conversion. In yet other embodiments, the bisulfite-converted nucleotide sequences of the disclosure also include nucleotide sequences that will hybridize under highly stringent conditions to any of the nucleotide sequences having at least 80%, 85%, 87%, 90%, 91%, 92%, 93%, 94%, 95%, 96%, 97%, 98%, 99% or 100% identity to any of SEQ ID NOs: 1-1760.

In further aspects, the application provides methods for producing such bisulfite-converted nucleotide sequences, for example, the application provides methods for treating a nucleotide sequence with a bisulfite agent such that the unmethylated cytosine bases are converted to a different nucleotide base such as a uracil.

In yet other aspects, the application provides oligonucleotide primers for amplifying a region within the nucleic acid sequence of any of the nucleotide sequences having at least 80%, 85%, 87%, 90%, 91%, 92%, 93%, 94%, 95%, 96%, 97%, 98%, 99% or 100% identity to any of SEQ ID NOs: 1-1760. In certain aspects, a pair of the oligonucleotide primers can be used in a detection assay, such as the HpaII assay. In certain aspects, primers used in an MSP reaction can specifically distinguish between methylated and non-methylated DNA.

The primers of the disclosure have sufficient length and appropriate sequence so as to provide specific initiation of amplification nucleic acids. Primers of the disclosure are designed to be “substantially” complementary to each strand of the nucleic acid sequence to be amplified. While exemplary primers are provided as SEQ ID NOs: 871-984, 1525-1550, 1689-1696 or 1751-1762, it is understood that any primer(s) that hybridizes with any of the bisulfite-converted sequences disclosed herein are included within the scope of this disclosure and is useful in the method of the disclosure for detecting methylated nucleic acid, as described. Similarly, it is understood that any primer(s) that would serve to amplify a methylation-sensitive restriction site or sites within the differentially methylated region of any of the informative loci disclosed herein, or fragments or complements thereof, are included within the scope of this disclosure and is useful in the method of the disclosure for detecting nucleic methylated nucleic acid, as described.

The oligonucleotide primers of the disclosure may be prepared by using any suitable method, such as conventional phosphotriester and phosphodiester methods or automated embodiments thereof. In one such automated embodiment, diethylphosphoramidites are used as starting materials and may be synthesized as described by Beaucage, et al. (Tetrahedron Letters, 22:1859-1862, 1981). One method of synthesizing oligonucleotides on a modified solid support is described in U.S. Pat. No. 4,458,066.

In some embodiments, the disclosure provides for primers for amplifying any of the informative loci sequences, or fragments or complements thereof, disclosed herein. In some embodiments, the disclosure provides for primers having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 consecutive nucleotides of any one or more of the sequences having at least 80%, 85%, 90%, 91%, 92%, 93%, 94%, 95%, 96%, 97%, 98%, 99%, or 100% identity to any of SEQ ID NOs: 871-984, 1525-1550, 1689-1696 or 1751-1762. In some embodiments, the primers comprise a sequence having at least 80%, 85%, 90%, 91%, 92%, 93%, 94%, 95%, 96%, 97%, 98%, 99%, or 100% identity to any of SEQ ID NOs: 871-984, 1525-1550, 1689-1696 or 1751-1762, or fragments or reverse complements thereof. In particular embodiments, the disclosure provides for primers having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 consecutive nucleotides of any one or more of the sequences having at least 80%, 85%, 90%, 91%, 92%, 93%, 94%, 95%, 96%, 97%, 98%, 99%, or 100% identity to any of SEQ ID NOs: 1525-1550, 1689-1696 or 1751-1762. In some embodiments, the primers comprise a sequence having at least 80%, 85%, 90%, 91%, 92%, 93%, 94%, 95%, 96%, 97%, 98%, 99%, or 100% identity to any of SEQ ID NOs: 1525-1550, 1689-1696 or 1751-1762. In particular embodiments, the disclosure provides for primers having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 consecutive nucleotides of any one or more of the sequences having at least 80%, 85%, 90%, 91%, 92%, 93%, 94%, 95%, 96%, 97%, 98%, 99%, or 100% identity to any of SEQ ID NOs: 1689-1696 or 1751-1762. In some embodiments, the primers comprise a sequence having at least 80%, 85%, 90%, 91%, 92%, 93%, 94%, 95%, 96%, 97%, 98%, 99%, or 100% identity to any of SEQ ID NOs: 1689-1696 or 1751-1762.

In some embodiments, the disclosure provides for nucleotide sequences amplified using any of the primer sequences disclosed herein. In some embodiments, the disclosure provides for amplicons that were generated using any one or more primer having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 consecutive nucleotides of any one or more of the sequences having at least 80%, 85%, 90%, 91%, 92%, 93%, 94%, 95%, 96%, 97%, 98%, 99%, or 100% identity to any of SEQ ID NOs: 871-984, 1525-1550, 1689-1696, or 1751-1762. In some embodiments, the amplicons comprise a nucleotide sequence that is at least 80%, 85%, 90%, 91%, 92%, 93%, 94%, 95%, 96%, 97%, 98%, 99%, or 100% identical to any of the sequences of SEQ ID NOs: 1099-1326, 1551-1628, or 1697-1720, or any fragments or complements thereof. In some embodiments, the amplicons comprise a nucleotide sequence that is at least 80%, 85%, 90%, 91%, 92%, 93%, 94%, 95%, 96%, 97%, 98%, 99%, or 100% identical to any of the sequences of SEQ ID NOs: 1099-1236, 1577-1628, or 1705-1720, or any fragments or complements thereof. In particular embodiments, the amplicons comprise a nucleotide sequence that is at least 80%, 85%, 90%, 91%, 92%, 93%, 94%, 95%, 96%, 97%, 98%, 99%, or 100% identical to any of the sequences of SEQ ID NOs: 1697-1720, or any fragments or complements thereof. In particular embodiments, the amplicons comprise a nucleotide sequence that is at least 80%, 85%, 90%, 91%, 92%, 93%, 94%, 95%, 96%, 97%, 98%, 99%, or 100% identical to any of the sequences of SEQ ID NOs: 1705-1720, or any fragments or complements thereof.

A fragment of any of the nucleotide sequences disclosed herein may be of any length, so long as the methylation status of at least a portion of that nucleotide sequence may be determined. In some embodiments, the nucleotide sequence is at least 10, 15, 25, 30, 40, 50, 60, 70, 80, 90, 100, 110, 120, 130, 140, 150, 160, 170, 180, 190, 200, 250, 300, 350, 400, 450, 500, 600, 700, 800, 900, 1000, 1200, 1400, 1500, 1700, or 2000 nucleotides in length. In some embodiments, the nucleotide sequence is at least 10-2000, 10-1000, 10-500, 10-200, 10-150, 10-100, 10-50, 10-30, 10-25, 10-20, 50-2000, 50-1000, 50-500, 50-200, 50-150, 50-100, 80-2000, 80-1000, 80-500, 80-150, 80-100, 100-2000, 100-1000, 100-500, 100-200, or 100-150 nucleotides in length.

### IV. Assays and Drug Screening Methodologies

In certain aspects, the application provides assays and methods using any of the informative loci disclosed herein, or fragments or complements thereof, as molecular markers that distinguish between healthy cells and cancer cells. For example, in one embodiment, the application provides methods and assays using any of the informative loci disclosed herein, or fragments or complements thereof, as markers that distinguish between healthy cells and neoplasia cells. In other embodiments, the application provides methods and assays using any of the informative loci disclosed herein, or fragments or complements thereof, as markers that distinguish between healthy cells and cells derived from neoplasias of the lower gastrointestinal tract. In one aspect, a molecular marker of the invention is a differentially methylated sequence of an informative locus.

In certain embodiments, the application provides assays for detecting differentially methylated nucleotide sequences. Thus, a differentially methylated nucleotide sequence, in its methylated state, can serve as a target for detection using various methods described herein and the methods that are well within the purview of the skilled artisan in view of the teachings of this application.

In certain embodiments, the disclosure provides for a method of assessing the methylation status of any individual nucleotide sequence disclosed herein. In some embodiments, the disclosure provides for a method of assessing the methylation of a panel of nucleotide sequences disclosed herein. In some embodiments, the panel comprises any one or more of the following combinations of sequences: 1) UnUp62 and UnUp229; 2) UnUp62, UnUp100, UnUp106, UnUp177, UnUp207, UnUp229 and UnUp307; 3) UnUp106 and UnUp146; 4) UnUp280 and UnUp307; 5) UnUp254 and UnUp307; 6) UnUp146 and UnUp254; 7) UnUp177 and UnUp307; 8) UnUp146 and UnUp307; 9) UnUp106 and UnUp307; 10) UnUp106, UnUp177 and UnUp307; 11) UnUp106, UnUp254, and UnUp307; 12) UnUp106, UnUp280 and UnUp307; 13) UnUp177, UnUp254 and UnUp307; 14) UnUp177, UnUp280 and UnUp307; 15) UnUp106, UnUp146, UnUp280 and UnUp307; 16) UnUp106, UnUp146, UnUp254 and UnUp307; 17) UnUp146, UnUp177, UnUp254 and UnUp307; or 18) UnUp106, UnUp207 and UnUp307. In particular embodiments, the panel comprises the following combination of sequences: UnUp106, UnUp146, UnUp207 and UnUp307.

In some embodiments, any of the informative loci disclosed herein is assessed in combination with an assessment of vimentin gene expression and/or methylation. In some embodiments, the disclosure provides for an assessment of the methylation status of any of the informative loci disclosed herein (or any combination of the informative loci disclosed herein) in combination with the methylation status of any of the vimentin nucleic acid sequences disclosed herein. In some embodiments, vimentin methylation is assessed in combination with assessing methylation of any of the following sequences or combinations of the following sequences: UnUp106, UnUp35, UnUp146, UnUp190, UnUp207, UnUp307, UnUp62, UnUp229, UnUp100, UnUp177, UnUp280 or UnUp254. In some embodiments, vimentin methylation is assessed in combination with assessing methylation of any of the following sequences or combinations of the following sequences: UnUp106, UnUp146, UnUp207 and UnUp307. In some embodiments, vimentin methylation is assessed in combination with assessing methylation of UnUp146.

In certain aspects, such methods for detecting methylated nucleotide sequences are based on treatment of genomic DNA with a chemical compound which converts non-methylated C, but not methylated C (i.e., 5 mC), to a different nucleotide base. One such compound is sodium bisulfite, which converts C, but not 5 mC, to U. Methods for bisulfite treatment of DNA are known in the art (Herman, et al., 1996, Proc Natl Acad Sci USA, 93:9821-6; Herman and Baylin, 1998, Current Protocols in Human Genetics, N. E. A. Dracopoli, ed., John Wiley & Sons, 2:10.6.1-10.6.10; U.S. Pat. No. 5,786,146). To illustrate, when a DNA molecule that contains unmethylated C nucleotides is treated with sodium bisulfite to become a compound-converted DNA, the sequence of that DNA is changed (C→U). Detection of the U in the converted nucleotide sequence is indicative of an unmethylated C.

The different nucleotide base (e.g., U) present in compound-converted nucleotide sequences can subsequently be detected in a variety of ways. In a preferred embodiment, the present invention provides a method of detecting U in compound-converted DNA sequences by using “methylation sensitive PCR” (MSP) (see, e.g., Herman. et al., 1996, Proc. Natl. Acad. Sci. USA, 93:9821-9826; U.S. Pat. No. 6,265,171; U.S. Pat. No. 6,017,704, U.S. Pat. No. 6,200,756). In MSP, one set of primers (i.e., comprising a forward and a reverse primer) amplifies the compound-converted template sequence if C bases in CpG dinucleotides within the DNA are methylated. This set of primers is called “methylation-specific primers.” Another set of primers amplifies the compound-converted template sequence if C bases in CpG dinucleotides within the 5′ flanking sequence are not methylated. This set of primers is called “unmethylation-specific primers.”

In MSP, the reactions use the compound-converted DNA from a sample in a subject. In assays for methylated DNA, methylation-specific primers are used. In the case where C within CpG dinucleotides of the target sequence of the DNA are methylated, the methylation-specific primers will amplify the compound-converted template sequence in the presence of a polymerase and an MSP product will be produced. If C within CpG dinucleotides of the target sequence of the DNA is not methylated, the methylation-specific primers will not amplify the compound-converted template sequence in the presence of a polymerase and an MSP product will not be produced.

It is often also useful to run a control reaction for the detection of unmethylated DNA. The reactions uses the compound-converted DNA from a sample in a subject and unmethylation-specific primers are used. In the case where C within CpG dinucleotides of the target sequence of the DNA are unmethylated, the unmethylation specific primers will amplify the compound-converted template sequence in the presence of a polymerase and an MSP product will be produced. If C within CpG dinucleotides of the target sequence of the DNA is methylated, the unmethylation-specific primers will not amplify the compound-converted template sequence in the presence of a polymerase and an MSP product will not be produced. Note that a biologic sample will often contain a mixture of both neoplastic cells that give rise to a signal with methylation specific primers, and normal cellular elements that give rise to a signal with unmethylation-specific primers. The unmethylation specific signal is often of use as a control reaction, but does not in this instance imply the absence of neoplasia as indicated by the positive signal derived from reactions using the methylation specific primers.

Primers for a MSP reaction are derived from the compound-converted template sequence. Herein, “derived from” means that the sequences of the primers are chosen such that the primers amplify the compound-converted template sequence in a MSP reaction. Each primer comprises a single-stranded DNA fragment which is at least 8 nucleotides in length. Preferably, the primers are less than 50 nucleotides in length, more preferably from 15 to 35 nucleotides in length. Because the compound-converted template sequence can be either the Watson strand or the Crick strand of the double-stranded DNA that is treated with sodium bisulfite, the sequences of the primers is dependent upon whether the Watson or Crick compound-converted template sequence is chosen to be amplified in the MSP. Either the Watson or Crick strand can be chosen to be amplified.

The compound-converted template sequence, and therefore the product of the MSP reaction, can, in some embodiments, be between 20 to 3000 nucleotides in length, between 50 to 500 nucleotides in length, or between 80 to 150 nucleotides in length. Preferably, the methylation-specific primers result in an MSP product of a different length than the MSP product produced by the unmethylation-specific primers.

A variety of methods can be used to determine if an MSP product has been produced in a reaction assay. One way to determine if an MSP product has been produced in the reaction is to analyze a portion of the reaction by agarose gel electrophoresis. For example, a horizontal agarose gel of from 0.6 to 2.0% agarose is made and a portion of the MSP reaction mixture is electrophoresed through the agarose gel. After electrophoresis, the agarose gel is stained with ethidium bromide. MSP products are visible when the gel is viewed during illumination with ultraviolet light. By comparison to standardized size markers, it is determined if the MSP product is of the correct expected size.

Other methods can be used to determine whether a product is made in an MSP reaction. One such method is called “real-time PCR.” Real-time PCR utilizes a thermal cycler (i.e., an instrument that provides the temperature changes necessary for the PCR reaction to occur) that incorporates a fluorimeter (i.e., an instrument that measures fluorescence). The real-time PCR reaction mixture also contains a reagent whose incorporation into a product can be quantified and whose quantification is indicative of copy number of that sequence in the template. One such reagent is a fluorescent dye, called SYBR Green I (Molecular Probes, Inc.; Eugene, Oreg.) that preferentially binds double-stranded DNA and whose fluorescence is greatly enhanced by binding of double-stranded DNA. When a PCR reaction is performed in the presence of SYBR Green I, resulting DNA products bind SYBR Green I and fluorescence. The fluorescence is detected and quantified by the fluorimeter. Such technique is particularly useful for quantification of the amount of the product in the PCR reaction. Additionally, the product from the PCR reaction may be quantitated in “real-time PCR” by the use of a variety of probes that hybridize to the product including TaqMan probes and molecular beacons. Quantitation may be on an absolute basis, or may be relative to a constitutively methylated DNA standard, or may be relative to an unmethylated DNA standard. In one instance the ratio of methylated derived product to unmethylated derived product may be constructed.

Methods for detecting methylation of the DNA according to the present disclosure are not limited to MSP, and may cover any assay for detecting DNA methylation. Another example method of detecting methylation of the DNA is by using “methylation-sensitive” restriction endonucleases. Such methods comprise treating the genomic DNA isolated from a subject with a methylation-sensitive restriction endonuclease and then using the restriction endonuclease-treated DNA as a template in a PCR reaction. Herein, methylation-sensitive restriction endonucleases recognize and cleave a specific sequence within the DNA if C bases within the recognition sequence are not methylated. If C bases within the recognition sequence of the restriction endonuclease are methylated, the DNA will not be cleaved. Examples of such methylation-sensitive restriction endonucleases include, but are not limited to HpaII, SmaI, SacII, EagI, BstUI, and BssHII. In this technique, a recognition sequence for a methylation-sensitive restriction endonuclease is located within the template DNA, at a position between the forward and reverse primers used for the PCR reaction. In the case that a C base within the methylation-sensitive restriction endonuclease recognition sequence is not methylated, the endonuclease will cleave the DNA template and a PCR product will not be formed when the DNA is used as a template in the PCR reaction. In the case that a C base within the methylation-sensitive restriction endonuclease recognition sequence is methylated, the endonuclease will not cleave the DNA template and a PCR product will be formed when the DNA is used as a template in the PCR reaction. Therefore, methylation of C bases can be determined by the absence or presence of a PCR product (Kane, et al., 1997, Cancer Res, 57: 808-11). No sodium bisulfite is used in this technique.

Yet another exemplary method of detecting methylation of the DNA is called the modified MSP, which method utilizes primers that are designed and chosen such that products of the MSP reaction are susceptible to digestion by restriction endonucleases, depending upon whether the compound-converted template sequence contains CpG dinucleotides or UpG dinucleotides.

Yet other methods for detecting methylation of the DNA include the MS-SnuPE methods. This method uses compound-converted DNA as a template in a primer extension reaction wherein the primers used produce a product, dependent upon whether the compound-converted template contains CpG dinucleotides or UpG dinucleotides (see e.g., Gonzalgo, et al., 1997, Nucleic Acids Res., 25:2529-31).

Another exemplary method of detecting methylation of the DNA is called COBRA (i.e., combined bisulfite restriction analysis). This method has been routinely used for DNA methylation detection and is well known in the art (see, e.g., Xiong, et al., 1997, Nucleic Acids Res, 25:2532-4). In this technique, methylation-sensitive restriction endonucleases recognize and cleave a specific sequence within the DNA if C bases within the recognition sequence are methylated. If C bases within the recognition sequence of the restriction endonuclease are not methylated, the DNA will not be cleaved.

Another exemplary method of detecting methylation of DNA requires hybridization of a compound converted DNA to arrays that include probes that hybridize to sequences derived from a methylated template.

Another exemplary method of detecting methylation of DNA includes precipitation of methylated DNA with antibodies that bind methylated DNA or with other proteins that bind methylated DNA, and then detection of DNA sequences in the precipitate. The detection of DNA could be done by PCR based methods, by hybridization to arrays, or by other methods known to those skilled in the art.

In certain embodiments, the disclosure provides methods that involve directly sequencing the product resulting from an MSP reaction to determine if the compound-converted template sequence contains CpG dinucleotides or UpG dinucleotides. Molecular biology techniques such as directly sequencing a PCR product are well known in the art. In some embodiments, the PCR products are sequenced by means of next generation sequencing.

In some embodiments, methylation of DNA may be measured as a percentage of total DNA. High levels of methylation may be 10-100% methylation, for example, 10%, 20%, 30%, 40%, 50%, 60%, 70%, 80%, 90%, or 100% methylation. Low levels of methylation may be 0%-9.99% methylation, for example, 0%, 0.1%, 0.5%, 1%, 2%, 3%, 4%, 5%, 6%, 7%, 8%, 9%, or 9.99%. At least some normal tissues, for example, normal colon samples, may not have any detectable methylation.

The skilled artisan will appreciate that the present disclosure is based in part, on the recognition that any one of the informative loci disclosed herein, or any of the fragments or complements thereof, may include nucleotide sequences that encode polypeptides that, for example, may function as a tumor suppressor gene. Accordingly, the application further provides methods for detecting such polypeptides in cell samples. In some embodiments, the disclosure provides detection methods by assaying such polypeptides so as to determine whether a patient has or does not have a disease condition. Further, such a disease condition may be characterized by decreased levels of such polypeptides. In certain embodiments, the disclosure provides methods for determining whether a patient is or is not likely to have cancer by detecting such polypeptides. In further embodiments, the disclosure provides methods for determining whether the patient is having a relapse or determining whether a patient's cancer is responding to treatment.

Optionally, such methods involve obtaining a quantitative measure of the protein in the sample. In view of this specification, one of skill in the art will recognize a wide range of techniques that may be employed to detect and optionally quantitate the presence of a protein. In some embodiments, a protein is detected with an antibody. In many embodiments, an antibody-based detection assay involves bringing the sample and the antibody into contact so that the antibody has an opportunity to bind to proteins having the corresponding epitope. In many embodiments, an antibody-based detection assay also typically involves a system for detecting the presence of antibody-epitope complexes, thereby achieving a detection of the presence of the proteins having the corresponding epitope. Antibodies may be used in a variety of detection techniques, including enzyme-linked immunosorbant assays (ELISAs), immunoprecipitations, Western blots. Antibody-independent techniques for identifying a protein may also be employed. For example, mass spectroscopy, particularly coupled with liquid chromatography, permits detection and quantification of large numbers of proteins in a sample. Two-dimensional gel electrophoresis may also be used to identify proteins, and may be coupled with mass spectroscopy or other detection techniques, such as N-terminal protein sequencing. RNA aptamers with specific binding for the protein of interest may also be generated and used as a detection reagent. Samples should generally be prepared in a manner that is consistent with the detection system to be employed. For example, a sample to be used in a protein detection system should generally be prepared in the absence of proteases. Likewise, a sample to be used in a nucleic acid detection system should generally be prepared in the absence of nucleases. In many instances, a sample for use in an antibody-based detection system will not be subjected to substantial preparatory steps. For example, urine may be used directly, as may saliva and blood, although blood will, in certain preferred embodiments, be separated into fractions such as plasma and serum.

In certain embodiments, a method of the disclosure comprises detecting the presence of an informative loci-expressed nucleic acid, such as an mRNA, in a sample. Optionally, the method involves obtaining a quantitative measure of the informative loci-expressed nucleic acid in the sample. In view of this specification, one of skill in the art will recognize a wide range of techniques that may be employed to detect and optionally quantitate the presence of a nucleic acid. Nucleic acid detection systems generally involve preparing a purified nucleic acid fraction of a sample, and subjecting the sample to a direct detection assay or an amplification process followed by a detection assay. Amplification may be achieved, for example, by polymerase chain reaction (PCR), reverse transcriptase (RT) and coupled RT-PCR. Detection of a nucleic acid is generally accomplished by probing the purified nucleic acid fraction with a probe that hybridizes to the nucleic acid of interest, and in many instances, detection involves an amplification as well. Northern blots, dot blots, microarrays, quantitative PCR, and quantitative RT-PCR are all well known methods for detecting a nucleic acid in a sample.

In certain embodiments, the disclosure provides nucleic acid probes that bind specifically to an informative loci nucleic acid. Such probes may be labeled with, for example, a fluorescent moiety, a radionuclide, an enzyme or an affinity tag such as a biotin moiety. For example, the TaqMan® system employs nucleic acid probes that are labeled in such a way that the fluorescent signal is quenched when the probe is free in solution and bright when the probe is incorporated into a larger nucleic acid.

Immunoscintigraphy using monoclonal antibodies directed at the informative loci may be used to detect and/or diagnose a cancer. For example, monoclonal antibodies against the informative loci labeled with ⁹⁹Technetium, ¹¹¹Indium, ¹²⁵Iodine—may be effectively used for such imaging. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, preferably 1-10 millicuries, most often 2-5 millicuries are administered. Thus, compositions according to the present invention useful as imaging agents comprising a targeting moiety conjugated to a radioactive moiety comprise 0.1-100 millicuries, in some embodiments preferably 1-10 millicuries, in some embodiments preferably 2-5 millicuries, in some embodiments more preferably 1-5 millicuries.

In some embodiments, the disclosure provides for a device useful for detecting the methylation status of any of the nucleotide sequences, or fragments or complements thereof, disclosed herein. In some embodiments, the disclosure provides for a kit comprising components useful for detecting the methylation status of the nucleotide sequences, or fragments, or complements thereof, disclosed herein.

In certain embodiments, the present disclosure provides drug screening assays for identifying test compounds which potentiate the tumor suppressor function of polypeptides encoded by sequences located in the informative loci disclosed herein, or any of the fragments or complements thereof. In one aspect, the assays detect test compounds which potentiate the expression level of polypeptides encoded by sequences located in the informative loci disclosed herein, or any of the fragments or complements thereof. In another aspect, the assays detect test compounds which inhibit the methylation of DNA. In certain embodiments, drug screening assays can be generated which detect test compounds on the basis of their ability to interfere with stability or function of polypeptides encoded by sequences located in the informative loci disclosed herein, or any of the fragments or complements thereof.

A variety of assay formats may be used and, in light of the present disclosure, those not expressly described herein will nevertheless be considered to be within the purview of ordinary skill in the art. Assay formats can approximate such conditions as protein expression level, methylation status of nucleotide sequences, tumor suppressing activity, and may be generated in many different forms. In many embodiments, the disclosure provides assays including both cell-free systems and cell-based assays which utilize intact cells.

Compounds to be tested can be produced, for example, by bacteria, yeast or other organisms (e.g., natural products), produced chemically (e.g., small molecules, including peptidomimetics), or produced recombinantly. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In certain embodiments, test compounds identified from these assays may be used in a therapeutic method of treating cancer.

Still another aspect of the application provides transgenic non-human animals which express a gene located within any one of the informative loci disclosed herein, or any of the fragments or complements thereof, or which have had one or more of such genomic gene(s) disrupted in at least one of the tissue or cell-types of the animal.

In another aspect, the application provides an animal model for cancer, which has a mis-expressed allele of a gene located within any one of the informative loci disclosed herein, or any of the fragments or complements thereof. Such a mouse model can then be used to study disorders arising from mis-expression of genes located within any one of the informative loci listed in disclosed herein, or any of the fragments or complements thereof.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of the polypeptides. For example, excision of a target sequence which interferes with the expression of a recombinant gene can be designed to activate expression of that gene. This interference with expression of the protein can result from a variety of mechanisms, such as spatial separation of the gene from the promoter element or an internal stop codon. Moreover, the transgene can be made wherein the coding sequence of the gene is flanked recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

In an illustrative embodiment, either the cre/loxP recombinase system of bacteriophage P1 (Lakso et al., (1992) Proc. Natl. Acad. Sci. USA 89:6232-6236; Orban et al., (1992) Proc. Natl. Acad. Sci. USA 89:6861-6865) or the FLP recombinase system of Saccharomyces cerevisiae (O'Gorman et al., (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al., (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

### V. Subjects and Samples

In certain aspects, the invention relates to a subject suspected of having or has a cancer such as a neoplasia of the lower gastrointestinal tract (e.g., colorectal cancer). Alternatively, a subject may be undergoing routine screening and may not necessarily be suspected of having such a neoplasia (e.g., cancer). In a preferred embodiment, the subject is a human subject, and the neoplasia is colon neoplasia. In some embodiments, the colon neoplasia is colon cancer. In some embodiments, the cancer is Stage I, Stage II, Stage III, or Stage IV colon cancer. In some embodiments, the cancer is Stage I, Stage II, Stage III, or Stage IV rectal cancer.

Assaying for biomarkers discussed above in a sample from subjects not known to have, e.g., a neoplasia of the lower gastrointestinal tract can aid in diagnosis of such a neoplasia in the subject. To illustrate, detecting the methylation status of the nucleotide sequences by MSP can be used by itself, or in combination with other various assays, to improve the sensitivity and/or specificity for detecting, e.g., a neoplasia of the lower gastrointestinal tract. Preferably, such detection is made at an early stage in the development of cancer, so that treatment is more likely to be effective.

In some embodiments, an informative loci in a subject is considered “methylated” for the purposes of determining whether or not the subject is prone to developing and/or has developed a colon neoplasia if the loci is at least 10%, 20%, 30%, 40%, 50%, 60%, 70%, 80%, 90%, or 100% methylated. In some embodiments, a DNA sample from a subject is treated with bisulfite, and the resulting bisulfite sequence corresponds to any of the nucleotide sequences disclosed herein comprising a “Y” nucleotide. In some embodiments, if at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the Y residues of the bisulfite-converted sequence have a C, the sequence is considered “methylated” for the purposes of determining whether or not the subject is prone to developing and/or has developed a colon neoplasia. In some embodiments, a DNA sample from a subject is treated with bisulfite, and the resulting bisulfite sequence corresponds to any of the nucleotide sequences disclosed herein comprising a “Y” nucleotide. In some embodiments, if at least 10%, 20%, 30%, 40%, 50%, 60%, 70%, 80%, 90%, or 100% of the Y residues of the bisulfite-converted sequence have a C, the sequence is considered “methylated” for the purposes of determining whether or not the subject is prone to developing and/or has developed a colon neoplasia. In some embodiments, a subject is determined to be prone to developing and/or has developed a colon neoplasia if a certain number of “Y” nucleotides in a bisulfite converted sequence are cytosines. In some embodiments, the certain number is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the Y residues of the bisulfite-converted sequence. In some embodiments, the certain number is least 10%, 20%, 30%, 40%, 50%, 60%, 70%, 80%, 90%, or 100% of the Y residues of the bisulfite-converted sequence. In certain embodiments, a subject is determined to be prone to developing and/or has developed a colon neoplasia if a certain percentage of DNA molecules from a sample from a subject are determined to be “methylated,” as defined herein. In some embodiments, the certain percentage of DNA molecules is at least 10%, 20%, 30%, 40%, 50%, 60%, 70%, 80%, 90%, or 100% of the DNA molecules from the sample are determined to be “methylated.” In some embodiments, the percentage of methylated DNA molecules is determined using next-generation sequencing. Exemplary cut-offs of DNA methylation and DNA molecule percentages may be found in the Examples section provided herein.

In addition to diagnosis, assaying of a marker in a sample from a subject not known to have, e.g., a neoplasia of the lower gastrointestinal tract, can be prognostic for the subject (i.e., indicating the probable course of the disease). To illustrate, subjects having a predisposition to develop a neoplasia of the lower gastrointestinal tract may possess methylated nucleotide sequences. Assaying of methylated informative loci in a sample from subjects can also be used to select a particular therapy or therapies which are particularly effective against, e.g., a neoplasia of the lower gastrointestinal tract in the subject, or to exclude therapies that are not likely to be effective.

Assaying of methylated informative loci in samples from subjects that are known to have, or to have had, a cancer is also useful. For example, the present methods can be used to identify whether therapy is effective or not for certain subjects. One or more samples are taken from the same subject prior to and following therapy, and assayed for the informative loci markers. A finding that an informative locus is methylated in the sample taken prior to therapy and absent (or at a lower level) after therapy may indicate that the therapy is effective and need not be altered. In those cases where the informative locus is methylated in the sample taken before therapy and in the sample taken after therapy, it may be desirable to alter the therapy to increase the likelihood that the cancer will be reduced in the subject. Thus, the present method may obviate the need to perform more invasive procedures which are used to determine a patient's response to therapy.

Cancers frequently recur following therapy in patients with advanced cancers. In this and other instances, the assays of the invention are useful for monitoring over time the status of a cancer associated with silencing of genes located in any of the informative loci disclosed herein, or fragments or complements thereof. For subjects in whom a cancer is progressing, there can be no DNA methylation in some or all samples when the first sample is taken and then appear in one or more samples when the second sample is taken. For subjects in which cancer is regressing, DNA methylation may be present in one or a number of samples when the first sample is taken and then be absent in some or all of these samples when the second sample is taken.

Samples for use with the methods described herein may be essentially any biological material of interest. For example, a sample may be a bodily fluid sample from a subject, a tissue sample from a subject, a solid or semi-solid sample from a subject, a primary cell culture or tissue culture of materials derived from a subject, cells from a cell line, or medium or other extracellular material from a cell or tissue culture, or a xenograft (meaning a sample of a cancer from a first subject, e.g., a human, that has been cultured in a second subject, e.g., an immuno-compromised mouse). The term “sample” as used herein is intended to encompass both a biological material obtained directly from a subject (which may be described as the primary sample) as well as any manipulated forms or portions of a primary sample. A sample may also be obtained by contacting a biological material with an exogenous liquid, resulting in the production of a lavage liquid containing some portion of the contacted biological material. Furthermore, the term “sample” is intended to encompass the primary sample after it has been mixed with one or more additive, such as preservatives, chelators, anti-clotting factors, etc.

In certain embodiments, a bodily fluid sample is a blood sample. In this case, the term “sample” is intended to encompass not only the blood as obtained directly from the patient but also fractions of the blood, such as plasma, serum, cell fractions (e.g., platelets, erythrocytes, and lymphocytes), protein preparations, nucleic acid preparations, etc. In certain embodiments, a bodily fluid sample is a urine sample or a colonic effluent sample. In certain embodiments, a bodily fluid sample is a stool sample. In some embodiments, the bodily fluid may be derived from the stomach, for example, gastric secretions, acid reflux, or vomit. In other embodiments, the bodily fluid may be a fluid secreted by the pancreas or bladder. In other embodiments, the body fluid may be saliva or spit.

In certain embodiments, a tissue sample is a biopsy taken from the mucosa of the gastrointestinal tract. In other embodiments, a tissue sample is the brushings from, e.g., the colon of a subject.

A subject is preferably a human subject, but it is expected that the molecular markers disclosed herein, and particularly their homologs from other animals, are of similar utility in other animals. In certain embodiments, it may be possible to detect a biomarker described herein (e.g., DNA methylation or protein expression level) directly in an organism without obtaining a separate portion of biological material. In such instances, the term “sample” is intended to encompass that portion of biological material that is contacted with a reagent or device involved in the detection process.

In certain embodiments, DNA which is used as the template in an MSP reaction is obtained from a bodily fluid sample. Examples of preferred bodily fluids are blood, serum, plasma, a blood-derived fraction, stool, colonic effluent or urine. Other body fluids can also be used. Because they can be easily obtained from a subject and can be used to screen for multiple diseases, blood or blood-derived fractions are especially useful. For example, it has been shown that DNA alterations in colorectal cancer patients can be detected in the blood of subjects (Hibi, et al., 1998, Cancer Res, 58:1405-7). Blood-derived fractions can comprise blood, serum, plasma, or other fractions. For example, a cellular fraction can be prepared as a “buffy coat” (i.e., leukocyte-enriched blood portion) by centrifuging 5 ml of whole blood for 10 min at 800 times gravity at room temperature. Red blood cells sediment most rapidly and are present as the bottom-most fraction in the centrifuge tube. The buffy coat is present as a thin creamy white colored layer on top of the red blood cells. The plasma portion of the blood forms a layer above the buffy coat. Fractions from blood can also be isolated in a variety of other ways. One method is by taking a fraction or fractions from a gradient used in centrifugation to enrich for a specific size or density of cells.

DNA is then isolated from samples from the bodily fluids. Procedures for isolation of DNA from such samples are well known to those skilled in the art. Commonly, such DNA isolation procedures comprise lysis of any cells present in the samples using detergents, for example. After cell lysis, proteins are commonly removed from the DNA using various proteases. RNA is removed using RNase. The DNA is then commonly extracted with phenol, precipitated in alcohol and dissolved in an aqueous solution.

### VI. Therapeutic Methods

In some embodiments, the disclosure provides for a method of determining whether a subject has any one or more of the methylated informative loci disclosed herein that are indicative of the presence of a colon neoplasia (e.g. colon cancer), wherein if the subject is determined to have a colon neoplasia, the subject is treated with an agent that treats the colon neoplasia. In some embodiments, the disclosure provides for a method of treating a subject determined to have colorectal neoplasia (e.g., colorectal cancer). In some embodiments, the treatment of the colorectal neoplasia is surgery (e.g., colectomy, segmental resection, low anterior resection, and proctectomy with colo-anal anastomosis), radiation therapy (e.g., external beam radiation therapy, endocavitary radiation therapy, brachytherapy, radioembolization), and/or chemotherapy (e.g., 5-Fluorouracil (5-FU); Capecitabine (Xeloda®); Irinotecan (Camptosar®); Oxaliplatin (Eloxatin®), FOLFOX: 5-FU, leucovorin, and oxaliplatin; CapeOx: Capecitabine and oxaliplatin; 5-FU and leucovorin, FOLFOX: 5-FU, leucovorin, and oxaliplatin; FOLFIRI: 5-FU, leucovorin, and irinotecan; FOLFOXIRI (leucovorin, 5-FU, oxaliplatin, and irinotecan); CapeOx: Capecitabine and oxaliplatin; 5-FU and leucovorin; Capecitabine; Irinotecan, VEGF targeted drugs such as Bevacizumab (Avastin®) and ziv-aflibercept (Zaltrap®); EGFR targeted drugs such as Cetuximab (Erbitux®) and panitumumab (Vectibix®); kinase inhibitors such as Regorafenib (Stivarga®).

The terms “treatment”, “treating”, “alleviation” and the like are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

Treating a neoplasia (e.g., colorectal cancer) in a subject refers to improving (improving the subject's condition), alleviating, delaying or slowing progression or onset, decreasing the severity of one or more symptoms associated with a colon neoplasia. For example, treating a metaplasia or neoplasia includes any one or more of: reducing growth, proliferation and/or survival of metaplastic/neoplastic cells, killing metaplastic/neoplastic cells (e.g., by necrosis, apoptosis or autophagy), decreasing metaplasia/neoplasia size, decreasing rate of metaplasia/neoplasia size increase, halting increase in metaplasia/neoplasia size, improving ability to swallow, decreasing internal bleeding, decreasing incidence of vomiting, reducing fatigue, decreasing the number of metastases, decreasing pain, increasing survival, and increasing progression free survival.

### EXEMPLIFICATION

The invention now being generally described, it will be more readily understood by reference to the following examples, which are included merely for purposes of illustration of certain aspects and embodiments of the present invention, and are not intended to limit the invention.

### Example 1: Identification of Colorectal Cancer Informative Loci

Methylated informative loci were initially identified using the technique of reduced representation bisulfite sequencing (RRBS) in a discovery set of 41 Stage II-IV colon cancers and 25 matched normal colon tissues from 25 of these same patients.

Discovery data were initially analyzed for each individual CpG residue in the RRBS data set. Individual CpGs were considered methylated in colon cancer if they showed methylation in less than 10% of DNA sequence reads in all of the readable normal samples, where at least 4 normal samples were readable, where a readable normal sample had equal to or greater than 20 reads covering the CpG, and if 50% or more of the readable cancer samples demonstrated percent methylation at a level that was at least 20 percentage points greater than the methylation level of the most methylated normal tissue sample, where a readable cancer sample had equal to or greater than 10 reads covering the CpG. At least 6 readable and methylated cancer samples were required in order to include a CpG on the methylated in colon cancer list. Such methylated CpGs were then aggregated into patches or loci by grouping together methylated CpGs that were within 200 bp of one another. Patches may consist of 1 CpG up to any number of CpGs that meet the above criteria. Fifty methylated patches were identified that correspond to SEQ ID NOs: 1-100. As outlined below, in confirmatory studies, 4 best methylated patches were defined based on defining a window in which defining individual DNA sequencing reads as methylated or unmethylated showed robust differences between normal and cancer tissues.

Table 1 in columns A-AV is an excerpt of results from these experiments, including four loci having preferred characteristics. In Table 1, column A records names assigned to 4 best genomic patches defined as methylated in colon cancer by RRBS analysis. Column B gives the genomic coordinates of the genomic patches defined as methylated by the above criteria. Columns C and D provide the genomic sequences of these patches on the respective genomic (+) and (−) strands. Columns E and F disclose the bisulfite converted sequences of these corresponding patches, with column E providing the bisulfite converted sequence of the (+) strand and column F providing the bisulfite converted sequence of the (−) strand. C residues that may be methylated or unmethylated, and hence may be bisulfite converted to T (if unmethylated) or remain as a C (if methylated), are designated with a Y (where Y denotes C or T), and where, after bisulfite conversion, actual maintenance of a Y designated base as a C is scored as methylation at that base. Thus, the entries represent the group of all combinations of all sequences in which 0, 1, or more than one Y is converted to a T. The reverse complements of these sequences of columns E and F will be obvious to one of ordinary skill in the art and are also included by implication in this disclosure. Columns G and H disclose the bisulfite converted sequences of the fully methylated form of the corresponding patches (i.e. in which all Y bases in every of the entries of columns E and F respectively are retained as a C), with column G corresponding to the (+) strand and column H corresponding to the (−) strand. The reverse complements of these sequences of columns G and H will be obvious to one of ordinary skill in the art and are also included by implication in this disclosure. Column I discloses the genomic coordinates of the region of interest (ROI) that was used as a target for primer design. The ROI encompasses a preferred region of the patch of column B that was technically attractive for amplification. ROI regions were chosen by extending the patches of column B by 50-200 bp on either side, so as to accommodate either design of amplification primers or to include presumptively methylated bases not directly assayed. Columns J and K provide the genomic sequences of these expanded ROIs on the respective genomic (+) and (−) strands. Columns L and M disclose the bisulfite converted sequences of these ROI regions, with column L providing the bisulfite converted sequence of the (+) strand and column M providing the bisulfite converted sequence of the (−) strand. C residues that may be methylated or unmethylated, and hence may be bisulfite converted to T (if unmethylated) or remain as a C (if methylated), are designated with a Y (where Y denotes C or T), and where, after bisulfite conversion, actual maintenance of a Y designated base as a C is scored as methylation at that base. Thus, the entries represent the group of all combinations of sequences in which 0, 1, or more than one Y is converted to a T. The reverse complements of these sequences of columns L and M will be obvious to one of ordinary skill in the art and are also included by implication in this disclosure. Columns N and O disclose the bisulfite converted sequences of the fully methylated form of the corresponding patches (i.e. in which all Y bases in every of the entries of columns L and N respectively are retained as a C), with column N corresponding to the (+) strand and column O corresponding to the (−) strand. The reverse complements of these sequences of columns N and O will be obvious to one of ordinary skill in the art and are also included by in this description. Column P provides the genomic coordinates of any CpG island that overlaps the patch of column B, and that by implication may be methylated coordinately with the patch of column B. Columns Q and R provide the genomic sequences of these CpG islands on the respective genomic (+) and (−) strands. Columns S and T disclose the bisulfite converted sequences of these corresponding patches, with column S providing the bisulfite converted sequence of the (+) strand and column T providing the bisulfite converted sequence of the (−) strand. C residues that may be methylated or unmethylated, and hence may be bisulfite converted to T (if unmethylated) or remain as a C (if methylated), are designated with a Y (where Y denotes C or T), and where, after bisulfite conversion, actual maintenance of a Y designated base as a C is scored as methylation at that base. Thus, the entries represent the group of all combinations of sequences in which 0, 1, or more than one Y is converted to a T. The reverse complements of these sequences of columns S and T will be obvious to one of ordinary skill in the art and are also included by implication in this disclosure. Columns U and V disclose the bisulfite converted sequences of the fully methylated form of the corresponding patches (i.e. in which all Y bases in every of the entries of columns S and T respectively are retained as a C), with column U corresponding to the (+) strand and column V corresponding to the (−) strand. The reverse complements of these sequences of columns U and V will be obvious to one of ordinary skill in the art and are also included by implication in this disclosure. Column W provides the length of these CpG islands. Column X provides the identity of any genes on the (+) strand that overlaps the patch of column B. Column Y provides the identity of any genes on the (−) strand that overlaps the patch of column B. Column Z identifies the nearest gene on the plus strand that is 3′ (on the plus strand) of the patch in column B. Column AA provides the distance to the identified nearest gene. Column AB identifies the nearest gene on the minus strand that is 3′ (on the minus strand) of the patch in column B. Column AC provides the distance to the identified nearest gene on the (−) strand). Column AD discloses which genomic strand, (+) or (−), was used as the basis for bisulfite-specific amplification for confirmatory analysis by bisulfite sequencing. Columns AE and AF disclose the respective forward and reverse PCR primers that are bisulfite specific and methylation indifferent for use in amplifying the corresponding regions (amplicon1) for bisulfite sequencing. When more than one amplicon was used, Columns AG and AH disclose the respective forward and reverse PCR primers for the second amplicon (amplicon2). In these sequences in columns AE through AH, Y indicates a degenerate base in the primer where Y may be either C or T, and R indicates a degenerate base in the primer where R may be either A or G. Columns AI and AJ disclose the genomic coordinates of the first and second amplicons that were generated for confirmatory analysis by bisulfite sequencing. Columns AK and AL respectively provide the genomic sequence of the (+) strand and the (−) strand) of amplicon1. Columns AM and AN provide the sequences of the (+) and (−) strand of amplicon2, where second amplicon was utilized for confirmatory sequencing. Columns AO and AP disclose the bisulfite converted sequences of these corresponding amplicons, with column AO providing the bisulfite converted sequence of the (+) strand of Amplicon1, and column AP providing the bisulfite converted sequence of the (−) strand of Amplicon1. C residues that may be methylated or unmethylated, and hence may be bisulfite converted to T (if unmethylated) or remain as a C (if methylated), are designated with a Y (where Y denotes C or T), and where, after bisulfite conversion, actual maintenance of a Y designated base as a C is scored as methylation at that base. Thus, the entries represent the group of all combinations of sequences in which 0, 1, or more than one Y is converted to a T. The reverse complements of these sequences of columns AO, AP, AQ and AR will be obvious to one of ordinary skill in the art and are also included by implication in this disclosure. Columns AS and AT disclose the bisulfite converted sequences of the fully methylated form of the amplicon1 (i.e. in which all Y bases in every of the entries of columns L and N respectively are retained as a C), with column AS corresponding to the (+) strand and column AT corresponding to the (−) strand. The reverse complements of these sequences of columns AS and AT will be obvious to one of ordinary skill in the art and are also included by implication in this disclosure.

Confirmatory analysis of loci from Table 1 was then done by Next Generation sequencing of bisulfite DNAs amplified to generate amplicon1 of Table 1. This employed an expanded sample set of resected colon tissues comprising: 20 Stage II cancers and 20 matching normal colon tissues from the same individuals, 20 Stage IV cancers and 20 matching normal colon tissues from the same individuals. In addition, eight colon cancer cell lines, 4 corresponding to Stage II cancers, and 4 established from Stage IV tumors were included in confirmatory analysis, along with the DNA corresponding to 8 primary tumors matching these cell lines. The most preferred loci were identified based on defining for each locus an analysis window in which defining individual DNA sequencing reads within the window as methylated or unmethylated showed robust differences between normal and cancer tissues.

Column AW provides the genomic coordinates of an exemplary small window1 for the four preferred loci. The window1 denotes a smaller CpG dense area within the ROI (“Region of Interest”) of column I, that was technically attractive for amplification of small DNA fragments, and that showed the best sensitivity and specificity for distinguishing cancer from normal tissue. The smaller size of window1 makes this region advantageous for use in analysis of DNA from body fluids because DNA from such samples may be degraded to smaller fragment size. Column AX provides the genomic sequence of the (+) strand of window1, and column AY provides the genomic sequence of the (−) strand of window1. Columns AZ and BA provide the bisulfite converted sequences of window1, with column AZ corresponding to the (+) strand and column BA corresponding to the (−) strand of window1. C residues that may be methylated or unmethylated, and hence may be bisulfite converted to T (if unmethylated) or remain as a C (if methylated), are designated with a Y (where Y denotes C or T), and where, after bisulfite conversion, actual maintenance of a Y designated base as a C is scored as methylation at that base. Thus, the entries represent the group of all combinations of sequences in which 0, 1, or more than one Y is converted to a T. The reverse complements of these sequences of columns AZ and BA will be obvious to one of ordinary skill in the art and are also included by implication in this disclosure. Columns BB and BC provide the bisulfite converted sequences of the fully methylated form of the corresponding window1 sequences, with BB corresponding to AZ and BC corresponding to BA (i.e., in which all Y bases in every of the entries of column BB and BC are retained as a C). The reverse complements of these sequences of column BB and BC will be obvious to one of ordinary skill in the art and are also included by implication in this disclosure.

When more than one window was designed, column BD provides the genomic coordinates of the second window (window2) that was technically attractive for amplification of small DNA fragments, and that also showed best sensitivity and specificity for distinguishing cancer from normal tissue. Column BE provides the genomic sequence of the (+) strand of window2, and column BF provides the corresponding (−) strand sequence. Columns BG and BH provide the bisulfite converted sequence of the (+) and (−) strand of window2, with the column BG corresponding to the bisulfite-converted (+) strand, and column BH corresponding to bisulfite-converted (−) strand. C residues that may be methylated or unmethylated, and hence may be bisulfite converted to T (if unmethylated) or remain as a C (if methylated), are designated with a Y (where Y denotes C or T), and where, after bisulfite conversion, actual maintenance of a Y designated base as a C is scored as methylation at that base. Thus, the entries represent the group of all combinations of sequences in which 0, 1, or more than one Y is converted to a T. The reverse complements of these sequences of column BG and BH will be obvious to one of ordinary skill in the art and are also included by implication in this disclosure. Columns BI and BJ provide the bisulfite converted sequences of the fully methylated form of the corresponding window2 sequences, with BI corresponding to column BG, and BJ corresponding to column BH (i.e., in which all Y bases in every of the entries of column BG and BH are retained as a C). The reverse complements of these sequences of columns BI and BJ will be obvious to one of ordinary skill in the art and are also included by implication in this disclosure.

Columns BK and BL provide the respective forward and reverse PCR primers that are bisulfite specific and methylation indifferent for use in amplifying the corresponding small window regions (window1) for bisulfite sequencing. When more than one window was used, Columns BM and BN provide the respective forward and reverse PCR primers for the second window (window2). In these sequences in columns BK through BN, Y indicates a degenerate base in the primer where Y may be either C or T, and R indicates a degenerate base in the primer where R may be either A or G.

TABLE 1A

| | | | | | | G | H |
|---|---|---|---|---|---|---|---|
| | | | | E | F | Patch | Patch |
| | | C | | Patch | Patch | sequence | sequence |
| | | Patch | D | sequence | sequence | (+) strand- | (−) strand- |
| | | sequence | Patch | (+) | (−) strand- | BS | BS |
| | B | (+) | sequence | strand-BS | BS | converted, | converted, |
| | patch | strand | (−) strand | converted | converted | Methylated | Methylated |
| A | coordinates | (SEQ ID | (SEQ ID | (SEQ ID | (SEQ ID | (SEQ ID | (SEQ ID |
| Patch_ID | (hg19) | NO) | NO) | NO) | NO) | NO) | NO) |
| Un_Up_106 | chr6: 163834786-163834910 | 1629 | 1633 | 1637 | 1641 | 1645 | 1649 |
| Un_Up_146 | chr8: 97506549-97506612 | 1630 | 1634 | 1638 | 1642 | 1646 | 1650 |
| Un_Up_207 | chr12: 113494789-113494900 | 1631 | 1635 | 1639 | 1643 | 1647 | 1651 |
| Un_Up_307 | chr22: 39853283-39853292 | 1632 | 1636 | 1640 | 1644 | 1648 | 1652 |

TABLE 1B

| | | | | L | M | N | O |
|---|---|---|---|---|---|---|---|
| | | | | ROI | ROI | ROI | ROI |
| | | J | | sequence | sequence | sequence | sequence |
| | | ROI | K | (+) | (−) | (+) strand- | (−) strand- |
| | | sequence | ROI | strand- | strand- | BS- | BS- |
| | | (+) | sequence | BS- | BS- | converted, | converted, |
| | I | strand | (−) strand | converted | converted | Methylated | Methylated |
| | ROI coordinates, | (SEQ ID | (SEQ ID | (SEQ ID | (SEQ ID | (SEQ ID | (SEQ ID |
| A | (hg19) | NO) | NO) | NO) | NO) | NO) | NO) |
| Un_Up_106 | chr6: 163834539-163835189 | 1653 | 1657 | 1661 | 1665 | 1669 | 1673 |
| Un_Up_146 | chr8: 97506327-97506856 | 1654 | 1658 | 1662 | 1666 | 1670 | 1674 |
| Un_Up_207 | chr12: 113494613-113495060 | 1655 | 1659 | 1663 | 1667 | 1671 | 1675 |
| Un_Up_307 | chr22: 39853093-39853488 | 1656 | 1660 | 1664 | 1668 | 1672 | 1676 |

TABLE 1C

| | | | | | | U | |
|---|---|---|---|---|---|---|---|
| | | | | | | CpG_Island | V |
| | | | | S | T | (+) | CpG_Island |
| | | Q | | CpG_Island | CpG_Island | strand- | (−) strand- |
| | | CpG_Island | R | (+) strand- | (−) strand | BS- | BS- |
| | P | (+) | CpG_Island | BS- | BS- | converted- | converted- |
| | CpG | strand | (−) strand | converted | converted | Methylated | Methylated |
| A | island_chr: | (SEQ ID | (SEQ ID | (SEQ ID | (SEQ ID | (SEQ ID | (SEQ ID |
| Patch_ID | start-stop | NO) | NO) | NO) | NO) | NO) | NO) |
| Un_Up_106 | | | | | | | |
| Un_Up_146 | chr8: 97505747-97507607 | 1677 | 1679 | 1681 | 1683 | 1685 | 1687 |
| Un_Up_207 | chr12: 113494389-113495534 | 1678 | 1680 | 1682 | 1684 | 1686 | 1688 |
| Un_Up_307 | | | | | | | |

TABLE 1D

| | | X | Y | |
|---|---|---|---|---|
| | | onTARGET | onTARGET | Z |
| A | W | genes | genes | Nearest neighbor_GENES |
| Patch_IB | CpG_Island_length | (+strand) | (−strand) | (+strand) |
| Un_Up_106 | | | | QKI(NM_006775) |
| | | | | [quaking homolog KH domain |
| | | | | RNA binding |
| | | | | (mouse)]<chr6: 163835674-163999628>, |
| | | | | QKI(NM_206853) |
| | | | | [quaking homolog KH |
| | | | | domain RNA binding |
| | | | | (mouse)]<chr6: 163835674-163999628>, |
| | | | | QKI(NM_206854) |
| | | | | [quaking homolog KH |
| | | | | domain RNA binding |
| | | | | (mouse)]<chr6: 1638356 |
| Un_Up_146 | 1860 | SDC2(NM_002998) | | PGCP(NM_016134)[plasma |
| | | [syndecan 2] | | glutamate carboxy- |
| | | <chr8: 97505881-97624037> | | peptidase]<chr8: 97657498-98155722> |
| Un_Up_207 | 1145 | | | DTK1(NM_004416)[deltex |
| | | | | homolog 1 (Drosophila)] |
| | | | | <chr12: 113495661-113535833> |
| Un_Up_307 | | | | MGAT3(NM_002409) |
| | | | | [mannosyl (beta-1 4-)- |
| | | | | glycoprotein beta-1 4-N- |
| | | | | acetylglucosaminyltransferase] |
| | | | | <chr22: 39853324-39888199> |

| | AA | | AC |
|---|---|---|---|
| | Nearest | AB | Nearest |
| A | neighbor_distance | Nearest neighbor_GENES | neighbor_distance |
| Patch_IB | (+strand) | (−strand) | (−strand) |
| Un_Up_106 | 888 | LINC-PARK2-3(line- | 20554 |
| | | PARK2-3)<chr6: 163810451-163814232> | |
| Un_Up_146 | 150949 | LINC-MTERFD1(line- | 107904 |
| | | MTERFD1)<chr8: 97379635-97398645> | |
| Un_Up_207 | 872 | RPL6P27/RPL6/RPL6P19/ | 647346 |
| | | RPL6P10(NM_000970) | |
| | | [ribosomal protein L6 | |
| | | pseudogene 27; ribosomal | |
| | | protein L6 pseudogene 19; | |
| | | ribosomal protein L6; | |
| | | ribosomal protein L6 | |
| | | pseudogene | |
| | | 10]<chr12: 112842993-112847443>, | |
| | | RPL6P27/ | |
| | | RPL6/RPL6P19/RPL6P10 | |
| | | (NM_001024662)[ribosoma | |
| Un_Up_307 | 41 | RPL3/ | 137613 |
| | | LOC653881(NM_000967) | |
| | | [ribosomal protein L3; similar | |
| | | to 60S ribosomal | |
| | | protein L3 | |
| | | (L4)]<chr22: 39708886-39715670>, | |
| | | RPL3/ | |
| | | LOC653881(NM_001033853) | |
| | | [ribosomal protein L3; | |
| | | similar to 60S ribosomal | |
| | | protein L3 | |
| | | (L4)]<chr22: 39708886-39715670> | |

TABLE 1E

| | | AE | AF | | | | |
|---|---|---|---|---|---|---|---|
| | | Forward | Reverse | | | | |
| | AD | Primer | primer | AG | AH | AI | AJ |
| | Amplicon | Amplicon1 | Amplicon1 | Forward | Reverse | Amplicon1 | Amplicon2 |
| A | designed | (SEQ ID | (SEQ ID | Primer | primer | coordinates | coordinates |
| Patch_IB | against: | NO) | NO) | Amplicon2 | Amplicon2 | (hg19) | (hg19) |
| Un_Up_106 | (+) Strand | 1689 | 1693 | | | chr6: | |
| | | | | | | 163834751-163834941 | |
| Un_Up_146 | (−) Strand | 1690 | 1694 | | | chr8: | |
| | | | | | | 97506516-97506680 | |
| Un_Up_207 | (+) Strand | 1691 | 1695 | | | chr12: | |
| | | | | | | 113494734-113494933 | |
| Un_Up_307 | (+) Strand | 1692 | 1696 | | | chr22: | |
| | | | | | | 39853180-39853369 | |

TABLE 1F

| | AK | AL | | |
|---|---|---|---|---|
| | Amplicon1 | Amplicon1 | AM | |
| | sequence, | sequence, | Amplicon2 | AN |
| | (+) strand | (−) strand | sequence, | Amplicon2 |
| A | (SEQ ID | (SEQ | (+) | sequence, |
| Patch_ID | NO) | ID NO) | strand | (−) strand |
| Un_Up_106 | 1697 | 1701 | | |
| Un_Up_146 | 1698 | 1702 | | |
| Un_Up_207 | 1699 | 1703 | | |
| Un_Up_307 | 1700 | 1704 | | |

TABLE 1G

| | | | AS | AT |
|---|---|---|---|---|
| | AO | AP | Amplicon1 | Amplicon1 |
| | Amplicon1 | Amplicon1 | sequence, | sequence, |
| | sequence, | sequence, | (+) strand- | (−) strand- |
| | (+) strand- | (−) strand- | BS- | BS- |
| | BS- | BS- | converted, | converted, |
| | converted | converted | Methylated | Methylated |
| A | (SEQ ID | (SEQ ID | (SEQ ID | (SEQ ID |
| Patch_ID | NO) | NO) | NO) | NO) |
| Un_Up_106 | 1705 | 1709 | 1713 | 1717 |
| Un_Up_146 | 1706 | 1710 | 1714 | 1718 |
| Un_Up_207 | 1707 | 1711 | 1715 | 1719 |
| Un_Up_307 | 1708 | 1712 | 1716 | 1720 |

TABLE 1H

| | | | | | | BB | BC |
|---|---|---|---|---|---|---|---|
| | | | | AZ | BA | Best small | Best small |
| | | AX | AY | Best small | Best small | window1 (+) | window1 |
| | | Best small | Best small | window1 (+) | window1 | strand, BS- | (−) strand, BS- |
| | AW | window1 (+) | window1 (−) | strand, BS- | (−) strand, BS- | converted- | converted- |
| A | Best small window1 | strand (SEQ | strand (SEQ | converted (SEQ | converted (SEQ | Methylated | Methylated |
| Patch_ID | coordinates (hg19) | ID NO) | ID NO) | ID NO) | ID NO) | (SEQ ID NO) | (SEQ ID NO) |
| Un_Up_106 | chr6: 163834750-163834862 | 1721 | 1725 | 1729 | 1733 | 1737 | 1741 |
| Un_Up_146 | chr8: 97506522-97506632 | 1722 | 1726 | 1730 | 1734 | 1738 | 1742 |
| Un_Up_207 | chr12: 113494734-113494841 | 1723 | 1727 | 1731 | 1735 | 1739 | 1743 |
| Un_Up_307 | chr22: 39853251-39853365 | 1724 | 1728 | 1732 | 1736 | 1740 | 1744 |

TABLE 1I

| | | | | | | | BJ |
|---|---|---|---|---|---|---|---|
| | | | | | | BI | Best small |
| | | | | BG | BH | Best small | window2 |
| | | BE | BF | Best small | Best small | window2 (+) | (−) strand, |
| | BD | Best small | Best small | window2 (+) | window2 (−) | strand, BS- | BS- |
| | Best small | window2 | window2 | strand, BS- | strand, BS- | converted- | converted- |
| | window2 | (+) strand | (−) strand | converted | converted | Methylated | Methylated |
| A | coordinates | (SEQ ID | (SEQ ID | (SEQ ID | (SEQ ID | (SEQ | (SEQ |
| Patch_ID | (hg19) | NO) | NO) | NO) | NO) | ID NO) | ID NO) |
| Un_Up_106 | | | | | | | |
| Un_Up_146 | chr8: | 1745 | 1746 | 1747 | 1748 | 1749 | 1750 |
| | 97506528-97506643 | | | | | | |
| Un_Up_207 | | | | | | | |
| Un_Up_307 | | | | | | | |

TABLE 1J

| | BK | BL | BM | BN |
|---|---|---|---|---|
| | Small | Small | Small | Small |
| | window1 | window1 | window2 | window2 |
| | F primer | R primer | F primer | R primer |
| A | (SEQ ID | (SEQ ID | (SEQ ID | (SEQ ID |
| Patch_ID | NO) | NO) | NO) | NO) |
| Un_Up_106 | 1751 | 1755 | | |
| Un_Up_146 | 1752 | 1756 | 1759 | 1760 |
| Un_Up_207 | 1753 | 1757 | | |
| Un_Up_307 | 1754 | 1758 | | |

TABLE 2A

| A | B | C | D | E | F | G | H | I |
|---|---|---|---|---|---|---|---|---|
| Patch | VIM | Un_Up_35 | Un_Up_62 | Un_Up_100 | Un_Up_106 | Un_Up_146 | Un_Up_177 | Un_Up_190 |
| Number of | 10 | 23 | 31 | 19 | 20 | 12 | 19 | 13 |
| CpGs in | | | | | | | | |
| amplicon | | | | | | | | |
| CpG used | 10+ | 22+ | 22+ | 15+ | 18+ | 11+ | 17+ | 11+ |
| Cutoff 0.1 | 54% | 75% | 18% | 21% | 58% | 90% | 63% | 67% |
| Sensitivity | | | | | | | | |
| Cutoff 0.1 | 100% | 100% | 100% | 100% | 100% | 97% | 100% | 100% |
| Specificity | | | | | | | | |
| Cut-off | 97% | 92% | 100% | 97% | 97% | 92% | 97% | 92% |
| 0.01 | | | | | | | | |
| Specificity | | | | | | | | |

| | | | | | | O |
|---|---|---|---|---|---|---|
| | | | | | | 2 cleanest |
| | | | | | | markers |
| | | | L | | | together: |
| A | J | K | Un_Up_254 | M | N | Un-Up-62, |
| Patch | Un_Up_207 | Un_Up_229 | ampiicon1 | Un_Up_280 | Un_Up_307 | Un-Up_229 |
| Number of | 18 | 7 | 28 | 13 | 34 | |
| CpGs in | | | | | | |
| amplicon | | | | | | |
| CpG used | 15+ | 6+ | 22+ | 11+ | 25+ | |
| Cutoff 0.1 | 67% | 6% | 77% | 72% | 91% | 19% |
| Sensitivity | | | | | | |
| Cutoff 0.1 | 100% | 100% | 100% | 100% | 100% | 100% |
| Specificity | | | | | | |
| Cut-off | 97% | 100% | 95% | 95% | 97% | 100% |
| 0.01 | | | | | | |
| Specificity | | | | | | |

TABLE 2B

| | P | | | | | | | |
|---|---|---|---|---|---|---|---|---|
| | Combining | | | | | | | |
| | NEW | | | | | | | |
| | markers with | | | | | | | |
| | >=97% | | | | | | | |
| | specificity at | | | | | | | |
| | 0.01 cutoff | | | | | | | |
| | (Un_Up_62, | | | | | | | |
| A | Un_Up_100, | | | | | | | |
| Patch | Un_Up_106, | | | | | | | |
| Number of | Un_Up_177, | | | S | T | | | |
| CpGs in | Un_Up_207, | Q | R | Un_Up_254 | Un_Up_146 | U | V | W |
| amplicon | Un_Up_229, | Un_Up_106 | Un_Up_280 | amplicon1 | Un_Up_254 | Un_Up_177 | Un_Up_146 | Un_Up_106 |
| CpG used | Un_Up_307) | Un_Up_146 | Un_Up_307 | Un_Up_307 | amplicon1 | Un_Up_307 | Un_Up_307 | Un_Up_307 |
| Cutoff 0.1 | 96% | 94% | 94% | 94% | 94% | 94% | 94% | 94% |
| Sensitivity | | | | | | | | |
| Cutoff 0.1 | 100% | 98% | 100% | 100% | 98% | 100% | 98% | 100% |
| Specificity | | | | | | | | |
| Cut-off 0.01 | 88% | 93% | 93% | 93% | 88% | 95% | 90% | 95% |
| Specificity | | | | | | | | |

TABLE 2C

| A | | | | | | | AD | AE |
|---|---|---|---|---|---|---|---|---|
| Patch | | Y | | AA | | AC | Un_Up_106 | Un_Up_146 |
| Number of | X | Un_Up_106 | Z | Un_Up_177 | AB | Un_Up_106 | Un_Up_146 | Un_Up_177 |
| CpGs | Un_Up_106 | Un_Up_254 | Un_Up_106 | Un_Up_254 | Un_Up_177 | Un_Up_146 | Un_Up_254 | Un_Up_254 |
| amplicon | Un_Up_177 | amplicon1 | Un_Up_280 | amplicon1 | Un_Up_280 | Un_Up_280 | amplicon1 | amplicon1 |
| CpG used | Un_Up_307 | Un_Up_307 | Un_Up_307 | Un_Up_307 | Un_Up_307 | Un_Up_307 | Un_Up_307 | Un_Up_307 |
| Cutoff 0.1 | 96% | 94% | 96% | 96% | 94% | 96% | 96% | 96% |
| Sensitivity | | | | | | | | |
| Cutoff 0.1 | 100% | 100% | 100% | 100% | 100% | 98% | 98% | 98% |
| Specificity | | | | | | | | |
| Cut-off 0.01 | 93% | 90% | 90% | 90% | 90% | 85% | 85% | 85% |
| Specificity | | | | | | | | |

Table 2 describes the performance of amplicons of specific loci identified in this study and based on the analysis of the bisulfite sequencing data from the confirmatory data set. In Table 2, columns C-N disclose the performance of amplicons of specific loci. For each DNA sequence read across each amplicon the number of CpGs that were methylated was counted and the read was classified as methylated or unmethyled using cutoffs for the number of methylated CpGs on the amplicon. Row 3 lists the number of CpGs between the amplification primers for each of the amplicons. Row 4 lists the number of CpGs that need to be methylated on an individual read to count that read as methylated (e.g. for Un_Up_35 there are 23 CpG residues between the primers, and 22+ (meaning >=22) CpGs must be methylated on a read to score it as methylated.

Row 5 records the sensitivity for detecting colon tumors, using criteria in which a sample was detected if it demonstrated methylation in greater than 10% (0.1) of all DNA reads. Row 6 records the specificity of each amplicon for not detecting normal colon again using criteria in which a sample was detected if it demonstrated methylation in greater than 10% (0.1) of all DNA reads. Row 7 records the specificity of each amplicon for not detecting normal colon now using criteria in which a sample was detected if it demonstrated methylation in greater than 1% (0.01) of all DNA reads. As a comparator, column B provides the same data for detecting methylation in the Vimentin (VIM) locus amplified using primers disclosed in Li et al. (Li M, et al. (2009) Sensitive digital quantification of DNA methylation in clinical samples. Nat Biotechnol 27(9):858-863). Genomic coordinates for the VIM locus analyzed are chr10: 17271466-17271520, and the primers for amplifying the VIM locus had the sequences of SEQ ID NOs: 1761 and 1762. The VIM locus amplicon is similar in size to the windows selected in Table 1. Amplicons need not be used individually, but can be combined into panels for detection of colon neoplasias. Examples of such panels, and their associated performance statistics, are provided in columns O through AE that provide the markers in the panel and the sensitivity and specificity resulting from the marker combination.

The sensitivity for detection colon cancer (96%) is the same among the combinations shown of: 7 amplicons of column P, three combinations of 4 amplicons (columns AC-AE), and three of five combinations of 3 amplicons (columns X, Z, AA).

Specificity for not detecting normal colon (100%), at a detection cutoff of 10% of reads being methylated, is the same among the combinations shown of: 7 amplicons of column P, all combinations of 3 amplicons (columns X-AB), and some combinations of 2 amplicons (columns R, S, U, W). When specificity is determined using a cutoff of 1% of reads being methylated, then among amplicons having 96% sensitivity, the highest specificity is 93%, demonstrated by one combination of 3 amplicons (column X). When specificity is determined using a cutoff of 1% of reads being methylated, then among amplicons of 94% sensitivity, the highest specificity is 95%, demonstrated by the combination of 2 amplicons in columns U and W; among amplicons of 94% sensitivity, 93% specificity is demonstrated by three combinations of 2 amplicons (Table 2, columns Q, R, S).

Table 3 describes the performance of the small windows selected from Table 1 in the analysis of the confirmatory data set. In Table 3, columns E-I disclose the performance of the computationally-selected windows of Table 1 in the confirmatory data set. For each DNA sequence read across each window the number of CpGs that were methylated was counted and the read was classified as methylated or unmethyled using cutoffs for the number of methylated CpGs on the amplicon. Where more than 1 window was selected from the original amplicon, row 3 lists the window number corresponding to Table 1. Row 4 lists the number of CpGs that need to be methylated on an individual read to count that read as methylated (e.g for Un_Up_106 window1, 10+ (meaning >=10). CpGs must be methylated on a read to score it as methylated.

Row 5 records the sensitivity for detecting colon tumors, using criteria in which a sample was detected if it demonstrated methylation in greater than 10% (0.1) of all DNA reads. Row 6 records the specificity of each window for not detecting normal colon again using criteria in which a sample was detected if it demonstrated methylation in greater than 10% (0.1) of all DNA reads. Row 7 records the specificity of each window for not detecting normal colon now using criteria in which a sample was detected if it demonstrated methylation in greater than 1% (0.01) of all DNA reads. As a comparator, column D provides the same data for detecting methylation in the Vimentin (VIM) locus amplified using primers disclosed in Li et al. (Li M, et al. (2009) Sensitive digital quantification of DNA methylation in clinical samples. Nat Biotechnol 27(9):858-863). Genomic coordinates for the VIM locus analyzed are chr10: 17271466-17271520, and the primers for amplifying the VIM locus had the sequences of SEQ ID NOs: 1761 and 1762. The VIM locus amplicon is similar in size to the windows selected in Table 1.

Windows need not be used individually, but can be combined into panels for detection of colon neoplasia. An example of such a panel that was analyzed was Un-up-106, Un-up_207, Un-up_307, and the sensitivity and specificity performance statistics resulting from this marker combination is provided in Table 3, column J. The combination of the 3 windows provided in column J has 92% sensitivity for detection of colon cancer. Specificity for not detecting normal colon is 100%, at a detection cutoff of 10% of reads being methylated, and remains at 100% when specificity is determined using a cutoff of 1% of reads being methylated.

TABLE 3

| A | | | D | E | F | G | H | I | J |
|---|---|---|---|---|---|---|---|---|---|
| Patch | B | C | VIM | Un_up_106 | Un_up_146 | Un_up_146 | Un_up_207 | Un_up_307 | Panel |
| Window# | | | | 1 | 1 | 2 | 1 | 1 | |
| CpG used | | | 10+ | 10+ | 9+ | 8+ | 8+ | 12+ | |
| Cut-off = | Sensitivity | | 54% | 52% | 88% | 85% | 54% | 78% | 92% |
| 0.1 | for tumors | | | | | | | | |
| Cut-off = | Specificity | | 100% | 100% | 97% | 97% | 100% | 100% | 100% |
| 0.1 | for normals | | | | | | | | |
| Cutt-off = | Specificity | | 97% | 100% | 95% | 95% | 100% | 100% | 100% |
| 0.01 | for normals | | | | | | | | |

### Example 2: Validation of Colorectal Cancer Informative Loci

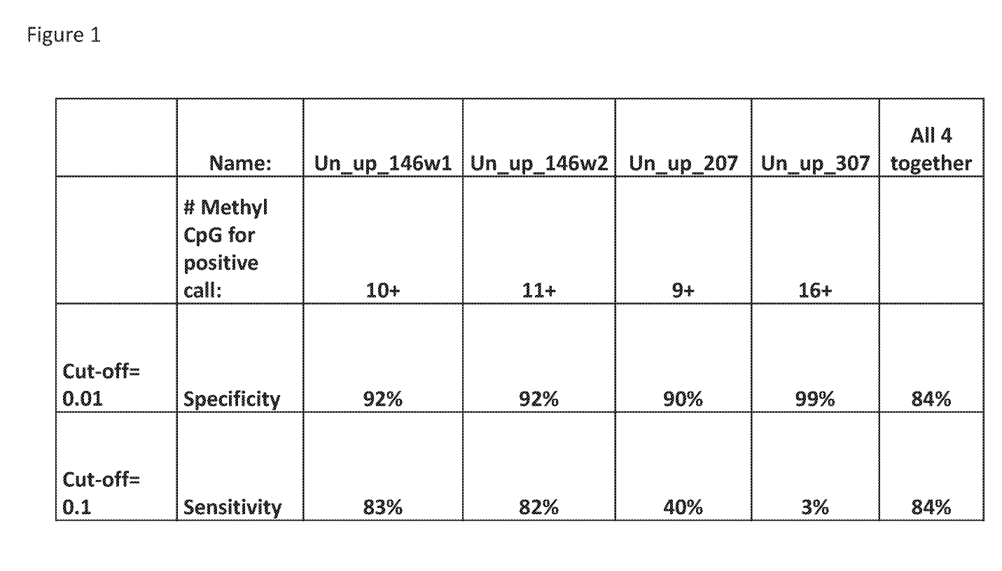

The specificity and sensitivity of three of the strongest methylated colon cancer markers previously identified (i.e., Un-up-146; Un-up-207; and Un-up-307) were determined using a fresh set of colon tumor and normal colon samples. A summary of the results are shown in FIG. 1. Marker Un-up-146 was tested for methylation within both window1 (w1) and window2 (w2). Methylation was assayed by amplifying the windows with the bisulfite specific and methylation indifferent PCR primers disclosed in the application. The number of CpGs within an amplicon that were required to be methylated to score a read as “methylated” is shown as the “# Methyl CpG for positive call”. Sensitivity was scored as the percent of tumors that showed greater than 10% of reads being methylated at each marker. Specificity was scored 1-false positive rate, where false positives were scored as normal samples that showed greater than 1% of reads being methylated at each marker.

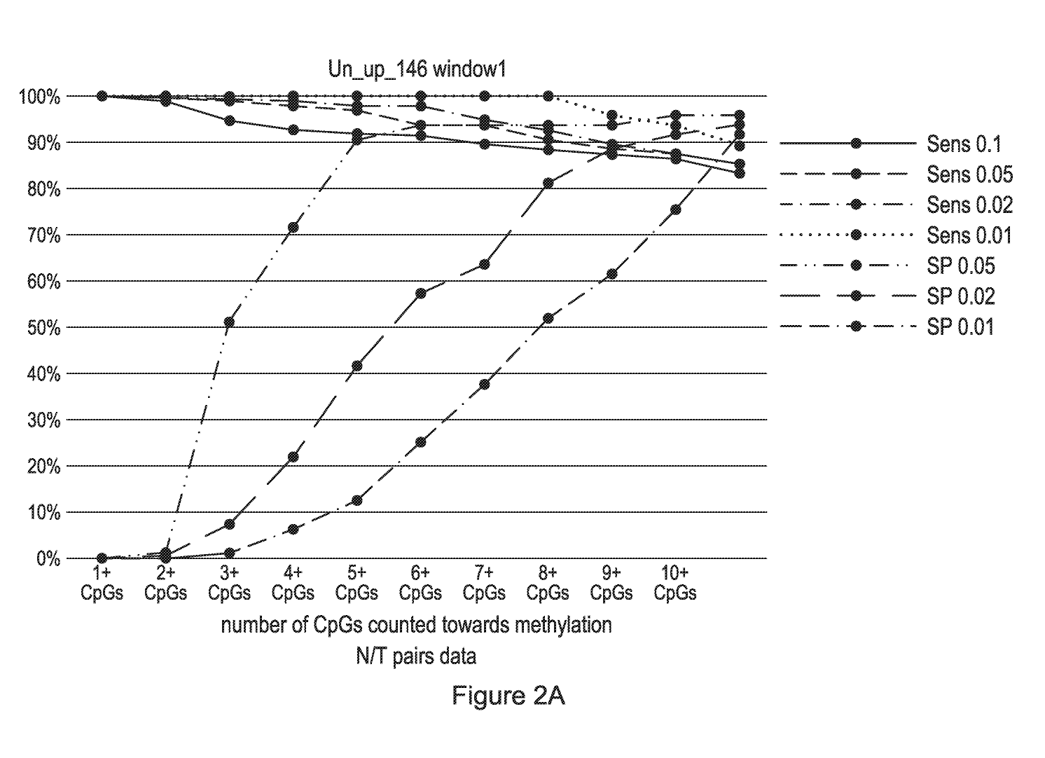

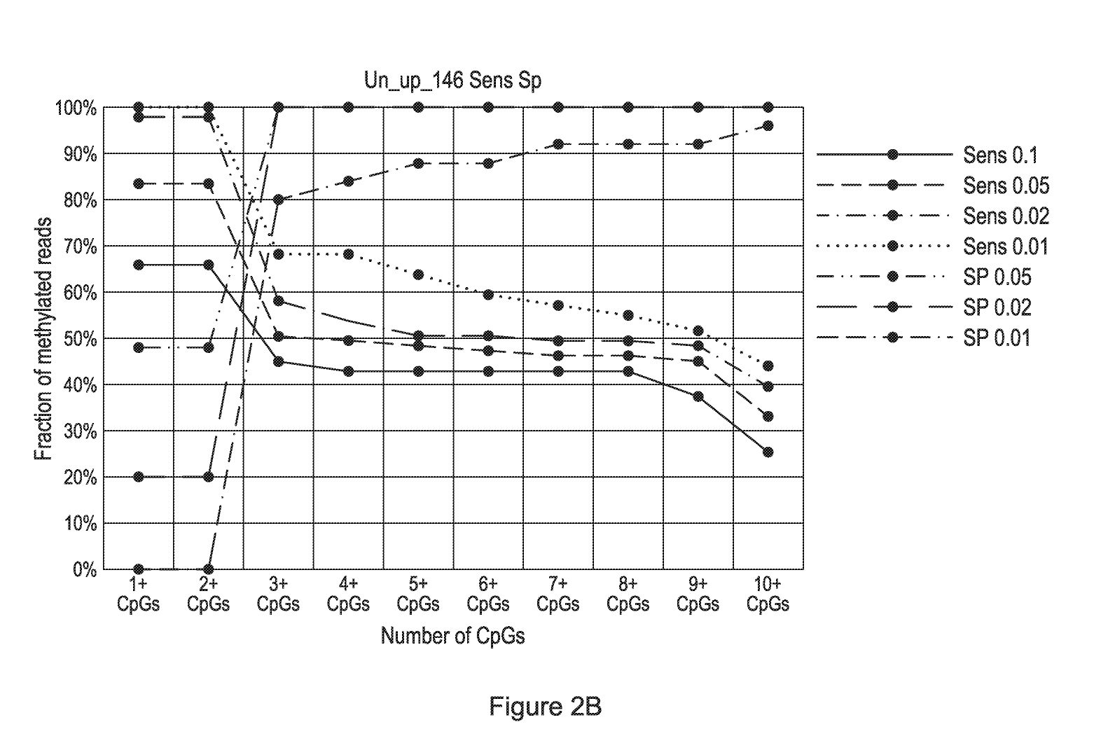

Marker Un-up-146 was further characterized using bisulfite sequencing in plasma versus colon samples from subjects having colon cancer or healthy control subjects. FIG. 2 shows graphs that show the sensitivity (Sens) for detecting a tumor sample or blood from a cancer patient, and the specificity (Sp) for not detecting a normal colon tissue or the blood from a control normal patient. FIG. 2A shows data for normal colon and colon tumors (N/T pairs). FIG. 2B shows data from plasma samples. Curves show the percent of samples detected (sensitivity) or not detected (specificity) when individual DNA reads that are called positive based on detection of methylation (i.e. retention of unconverted cytosine residues) at greater than or equal to the cutoff specified on the X-axis (e.g. 6+ designates a DNA read is termed methylated if greater than or equal to 6 CpG cytosines are detected as methylated in between the amplification primers). Curves show the percent of samples that are detected (sensitivity) or rejected (specificity) based on detecting a greater than or equal to percentage of DNA reads as being methylated (Y-axis). As demonstrated in FIGS. 2A and 2B, the plasma samples showed a lower methylation background than normal colon tissue when using cutoffs of 6+ CpG for calling a DNA read as methylated and 2% methylated reads for calling a sample as methylated is 100% specific in blood, but 57% specific in colon tissue. Un_Up_146 can be analyzed in plasma at a cut-off of 6+ CpG for calling a DNA read methylated and of 0.02 fraction of methylated reads for calling a sample as methylated.

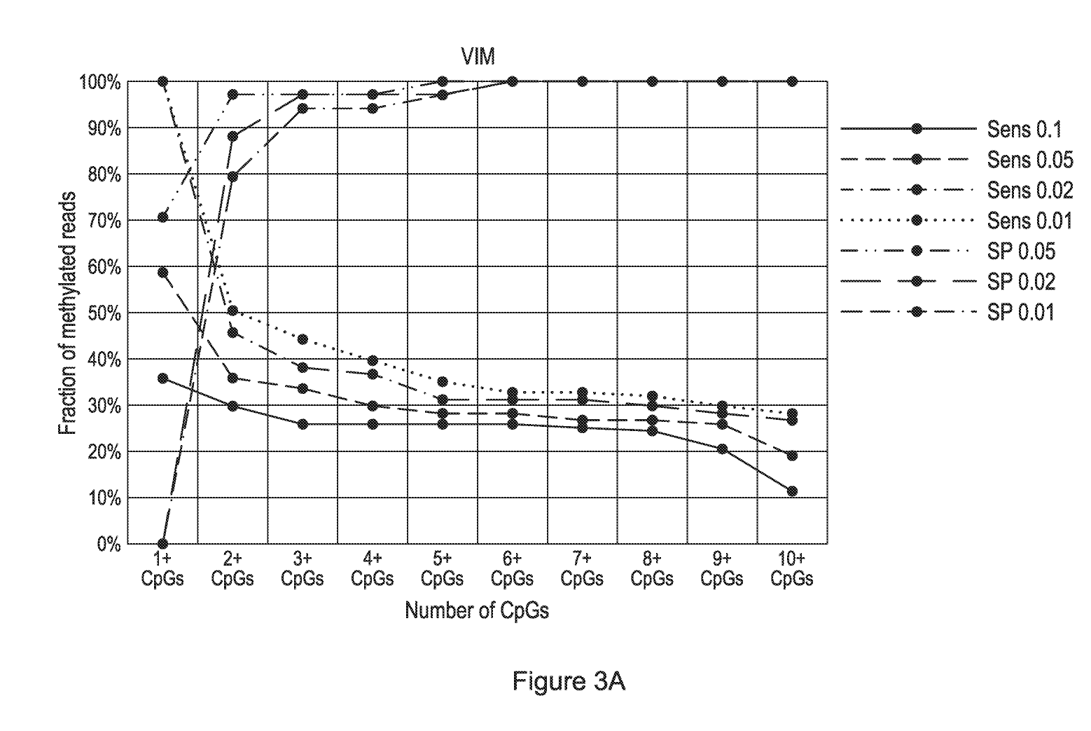

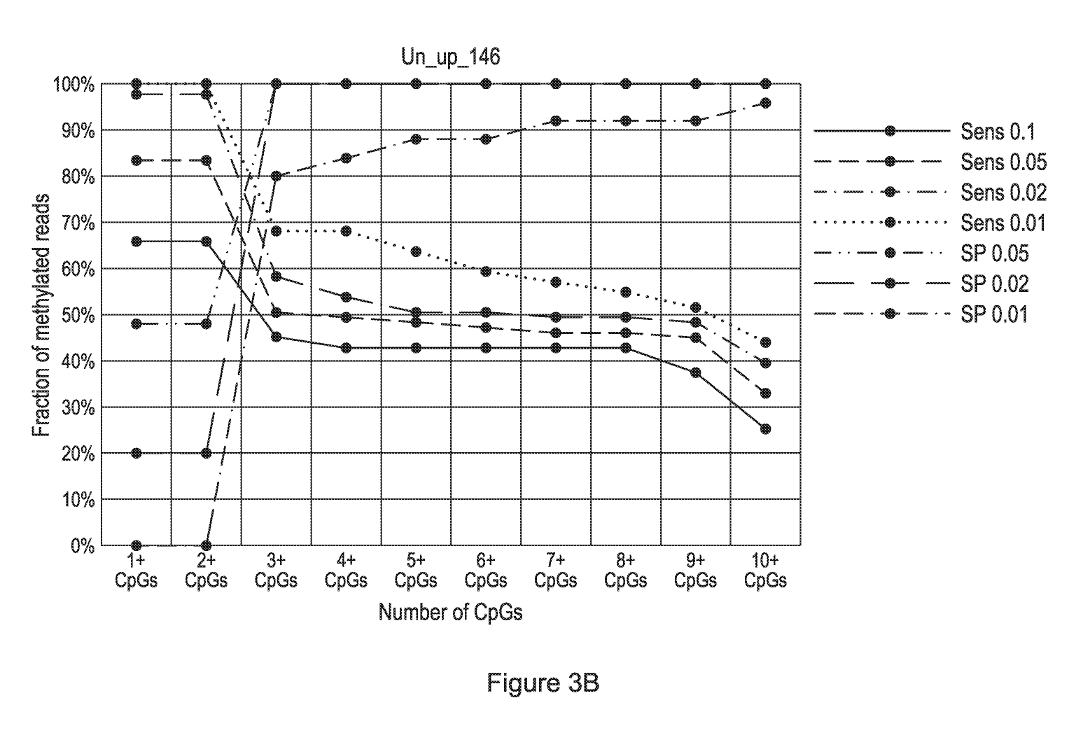

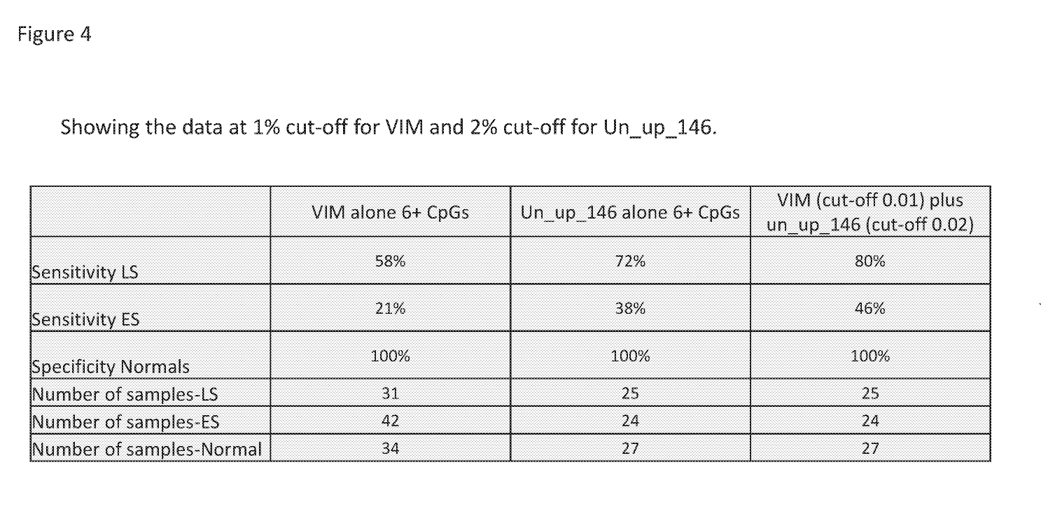

In an additional experiment, methylation of VIM was compared to methylation of Un_UpUI46 in the samples described in FIG. 2. As demonstrated in FIGS. 3A and 3B, Vim remained 100% specific in plasma at a cutoff of 6+ CpG for calling a DNA read as methylated and 1% methylated reads for calling a sample as methylated. Un_Up_146 remains 100% specific at a cutoff of 6+ CpG for calling a DNA read as methylated and 2% methylated reads for calling a sample as methylated. FIG. 4 provides a tabular summary of the sensitivity and specificity of the assay of plasma samples for Vim methylation and for Un_Up-146 methylation when the markers are analyzed either individually or in combination. Patients were further categorized as having either early stage (stage I or stage II) colon cancer, or as having late stage (stage III, stage IV, or metastatic recurrence) colon cancer. The combination of Vim plus Un_Up_146 methylation assays provides increased sensitivity for detection of individuals with early and with late stage colon cancers.

1-18. (canceled)

19. A method of treating a subject having colorectal cancer or neoplasia, comprising the step of treating the subject with chemotherapy, radiation therapy and/or with cancer resection or neoplasia resection; wherein said subject has been determined to DNA methylation as detected assay in a bisulfite converted DNA for retention of a cytosine base of one or more of the Y positions present in one or more of the nucleotide sequences having at least 90% identical to the sequence of any one or more of SEQ ID NOs: 101-200, 401-500, 691-780, 1099-1212, 1351-1374, 1423-1446, 1489-1506, 1577-1602, 1637-1644, 1661-1668, 1681-1684, 1705-1712, 1729-1736 or 1747-1748.

20. A bisulfite converted sequence comprising a nucleotide sequence having at least 90% identity to the sequence of any one or more of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750, and the reverse complements thereof, including all unique fragments of these sequences and their reverse complements.

21. A panel of bisulfite converted sequences selected from SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750, and the reverse complements thereof, including all unique fragments of these sequences and their reverse complements.

22. The panel of claim 21, wherein the panel corresponds to the combination of sequence regions comprising any one or more of the following combinations of sequences: 1) UnUp62 and UnUp229; 2) UnUp62, UnUp100, UnUp106, UnUp177, UnUp207, UnUp229 and UnUp307; 3) UnUp106 and UnUp146; 4) UnUp280 and UnUp307; 5) UnUp254 and UnUp307; 6) UnUp146 and
UnUp254; 7) UnUp177 and UnUp307; 8) UnUp146 and UnUp307; 9) UnUp106 and UnUp307; 10) UnUp106, UnUp177 and UnUp307; 11) UnUp106, UnUp254, and UnUp307; 12) UnUp106, UnUp280 and UnUp307; 13) UnUp177, UnUp254
and UnUp307; 14) UnUp177, UnUp280 and UnUp307; 15) UnUp106, UnUp146, UnUp280 and UnUp307; 16) UnUp106, UnUp146, UnUp254 and UnUp307; 17) UnUp146, UnUp177, UnUp254 and UnUp307; or 18) UnUp106, UnUp207 and UnUp307.

23. The panels of claim 21, wherein the panels correspond to the combination of sequence regions corresponding to UnUp106, UnUp146, UnUp207, and UnUp307.

24. The panel of claim 22, wherein the panel further comprises the vimentin sequence.

25. The panel of claim 24, wherein the panel corresponds to the combination of sequence regions corresponding to vimentin and UnUp146.

26. An oligonucleotide primer or probe that hybridizes to any of the sequences of claim 20.

27. (canceled)

28. The primers or probes of claim 26, wherein such primers or probes comprise any sequence having at least 90% sequence identity to any one or more of SEQ ID NOs: 1525-1550, 1689-1696 or 1751-1760.

29-35. (canceled)

36. A method for selecting an individual to undergo a diagnostic procedure to determine the presence of colon neoplasia, colon adenoma, colon cancer, or recurrence of colon cancer within the body, by obtaining a biological sample from an individual, and determining the presence in DNA from that sample of DNA methylation as detected assay in a bisulfite converted DNA for retention of a cytosine base present in any one or more of the nucleotide sequences having at least 90% identical to the sequence of any one or more of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750.

37. (canceled)

38. A method for selecting an individual to undergo a treatment for colon neoplasia, colon adenoma, colon cancer, or recurrence of colon cancer, by obtaining a biological sample from an individual, and determining the presence in DNA from that sample of DNA methylation as detected assay in a bisulfite converted DNA for retention of a cytosine base present in any one or more of the nucleotide sequences having at least 90% identical to the sequence of any one or more of SEQ ID NOs: 101-300, 401-600, 691-870, 1099-1326, 1351-1398, 1423-1470, 1489-1524, 1577-1628, 1637-1652, 1661-1688, 1681-1696, 1705-1720, 1729-1744, or 1747-1750.

39-40. (canceled)

41. The method of claim 36, wherein the bisulfite converted sequences are detected using any of: DNA sequencing, next generation sequencing, methylation specific PCR, methylation specific PCR combined with a fluorogenic hybridization probe, real time methylation specific PCR.

42. (canceled)

43. The method of claim 36, wherein the biological sample is a tissue sample or a body fluid.

44. (canceled)

45. The method of claim 43, wherein the body fluid is blood, saliva, spit, stool, or urine or a colonic lavage.

46. (canceled)

47. A method for determining the response of an individual with colorectal cancer to therapy by detection in a body fluid of methylation in any one or more of the nucleotide sequences having at least 90% identical to the sequence of any one or more of SEQ ID NOs: 101-300, 401-600, 691-870, 1099-1326, 1351-1398, 1423-1470, 1489-1524, 1577-1628, 1637-1652, 1661-1676, 1681-1688, 1705-1720, 1729-1744, or 1747-1750; wherein increasing levels of methylation over time are indicative of disease progression and a need for change to a new therapy, and wherein absence of increase in levels of methylation over time or decrease in levels of methylation over time are indicative that change in therapy is not required.

48. The method of claim 47, wherein DNA methylation is detected by bisulfite converting DNA from a body fluid and detecting the presence of any of the bisulfite converted DNA sequences of claim 20.

49. A bisulfite-converted nucleotide sequence comprising a sequence having at least 80%, 85%, 90%, 91%, 92%, 93%, 94%, 95%, 96%, 97%, 98%, 99% or 100% identity to any of the following sequences SEQ ID NO: 1705-1720, 1577-1628, 1729-1744, and 1747-1750.

50. The bisulfite-converted nucleotide sequence of claim 49, wherein the sequence comprises a sequence having at least 80%, 85%, 90%, 91%, 92%, 93%, 94%, 95%, 96%, 97%, 98%, 99% or 100% identity to any of the following sequences SEQ ID NO: 1705-1720.

51. The bisulfite-converted nucleotide sequence of claim 50, wherein the sequence comprises a sequence having at least 80%, 85%, 90%, 91%, 92%, 93%, 94%, 95%, 96%, 97%, 98%, 99% or 100% identity to 1706, 1710, 1714 or 1718.

52. (canceled)

53. A method of treating a subject having a colorectal neoplasia, comprising the step of treating the subject with chemotherapy, radiation therapy and/or with the resection of the neoplasia; and/or with ablation of the neoplasia; wherein said subject has been determined to have DNA methylation by assay in a bisulfite converted DNA for retention of a cytosine base of one or more of the Y positions present in one or more of the nucleotide sequences having at least 90% identity to the sequence of any one or more of: SEQ ID NOs: 101-300, 401-600, 691-870, 1099-1326, 1351-1398, 1423-1470, 1489-1524, 1577-1628, 1637-1652, 1661-1688, 1681-1696, 1705-1720, 1729-1744, or 1747-1750.

54. The method of claim 38, wherein the bisulfite converted sequences are detected using any of: DNA sequencing, next generation sequencing, methylation specific PCR, methylation specific PCR combined with a fluorogenic hybridization probe, real time methylation specific PCR.

55. The method of claim 38, wherein the biological sample is a tissue sample or a body fluid.

56. The method of claim 55, wherein the body fluid is blood, saliva, spit, stool, or urine or a colonic lavage.
